SEQUENCE LISTING (1 sequence)

SEQ ID NO: 1 - DNA, 102 bp, Homo sapiens
gagagggccctctgcctgctgcccgacctgctggtgccactctggaaagggcccaagactctctccccagggaagaatggggtcgtcaaagacgtttttgcc

(19) United States
(12) Patent Application Publication
(10) Pub. No.: US 2012/0264159 A1
(43) Pub. Date: Oct. 18, 2012

(54) SCREENING SYSTEM FOR MODULATORS OF HER2 MEDIATED TRANSCRIPTION AND HER2 MODULATORS IDENTIFED THEREBY

(75) Inventor: Christopher C. Benz, Novato, CA (US)

(73) Assignee: BUCK INSTITUTE FOR AGE RESEARCH, Novato, CA (US)

(21) Appl. No.: 12/726,071

(22) Filed: Mar. 17, 2010

Related U.S. Application Data
(62) Division of application No. 10/493,141, filed on Oct. 25, 2004, now abandoned.
(60) Provisional application No. 60/346,262, filed on Oct. 25, 2001.
(60) Provisional application No. 60/335,290, filed on Nov. 30, 2001.
(60) Provisional application No. 60/374,161, filed on Apr. 17, 2002.

(51) Int. Cl. C12Q 1/02

(52) U.S. Cl. 435/29

(57) ABSTRACT

This invention pertains to the development of a screening system to identify (screen for) HER2 promoter silencing agents. Such agents are expected to be of therapeutic value in the treatment of cancers characterized by HER2 amplification/upregulation. In addition, this invention pertains to the discovery that histone deacetylase (HDAC) inhibitors like sodium butyrate and trichostatin A (TSA), in a time and dose dependent fashion can silence genomically integrated and/or amplified/overexpressing promoters, such as that driving the HER2/ErbB2/neu oncogene, resulting in inhibition of gene products including transcripts and protein, and subsequent production of tumor/cell growth inhibition, apoptosis and/or differentiation. In another embodiment, this invention provides novel SNPs associated with the coding region of the ERbB2 proto-oncogene. The SNPs are indicators for altered risk, for developing ErbB2-positive cancer in a mammal.

### CROSS-REFERENCE TO RELATED APPLICATIONS

This application claims priority to and benefit of U.S. Ser. No. 60/346,262 filed on Oct. 25, 2001, U.S. Ser. No. 60/374,161, filed on Apr. 17, 2002, and U.S. Ser. No. 60/335,290, filed on Nov. 30, 2001, all of which are incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No. CA36773, awarded by the National Institutes of Health The Government of the United States of America may have certain rights in this invention.

### FIELD OF THE INVENTION

This pertains, to the fields of gene regulation and oncology. In particular this invention provides novel screening systems for identifying test agents that modulate expression of the HER2 (neu/ErbB2) oncogene.

### BACKGROUND OF THE INVENTION

Amplification and/or transcriptional overexpression of the HER2 (neu/ErbB2) oncogene in primary tumors is a proven prognostic marker of breast cancer, correlating with more aggressive tumor growth, decrease in patient survival, and altered responses to radiation, hormone, and chemothereapy (Alamon et al. (1987 (Science, 235: 177-182; Hannna et all (1999) Mod. Pathol., 12(8): 827-834; Benz and Tripathy (2000) J. Woman's Cancer, 2: 33-40). Since the discovery of this oncogene in 1985, numerous studies have implicated activated HER in the pathogenesis of breast, ovarian, and other cancers (Benz and Tripathy (2000) J. Woman's Cancer, 2: 33-40). HER2 represents an ideal therapeutic target, encoding an epithelial cell surface receptor tyrosine kinase that is homogeneously overexpressed in cancer cells yet expressed at low levels in normal human tissue (Benz and Tripathy (2000) J. Woman's Cancer, 2: 33-40).

Encouragingly, the first anti-HER2 therapeutic agent, trastuzumab (Herceptin; Genentech, Inc.), a humanized monoclonal antibody, has recently received FDA approval following demonstration of its safety and efficacy in clinical trials (id.). However, only about 20% of HER2 overexpressing patients respond to single agent trastuzumab. Alternative therapeutic strategies are thus clearly required.

Since transcriptional upregulation of HER2 commonly accompanies (and may in fact predispose to) gene amplification, an alternative to targeting HER2 receptor function is to inhibit transcription from the 2-10 fold amplified HER2 gene copies in certain cancer cells. Preliminary experiments have provided proof-of-principle verification of several promoter-silencing strategies (Noonberg et al. (1994) Gene 149(1): 123-126; Noonberg et al. (1995) J. Invest. Med., 43(suppl 1): 177A; Noonberg et al. (1995) AACR, 36: 432, Scott et al (1998) AACR 39: 1229; Chang et al. (1997) AACR, 38: 2334; and reviewed in Scott et al. (2000) Oncogene 19: 6490-6502), however, effective HER2 promoter down regulating/silencing agents are still desired.

### SUMMARY OF THE INVENTION

This invention pertains to a novel screening system used to screen for agents that modulate (e.g. upregulate or downregulate) activity of the HER2 promoter. In general, the screening system comprises a cell comprising a reporter gene operably linked to a heterologous HER2/ErbB2 promoter, where the promoter and the reporter are stably integrated into the genome of the cell.

Thus, in one embodiment, this invention provides a method of screening for an agent that modulates activity of a HER2/ErbB2 promoter. The method involves providing a cell comprising a reporter gene operably linked to a heterologous HER2/ErbB2 promoter, where the promoter and reporter are stably integrated into the genome of the cell; contacting said the with a test agent; and detecting expression of the reporter gene where a change in expression of said reporter gene as compared to a control indicates that said test agent modulates activity of said HER2/ErbB2 promoter. In certain embodiments, the control is the same assay performed with said test agent at a different concentration (e.g. a lower concentration, the absence of the test agent, etc.). Preferred test agents include, but are not limited to test agents known to downregulate HER2/ErbB2 expression. In certain embodiments, the control is performed with, a histone deacetylase (HDAC) inhibitor (e.g. sodium butyrate, trichostatin A, etc.). In a particularly preferred embodiment, the HER2/ErbB2 promoter comprises one or more genomically integrated and transcriptionally active copies of the promoter-reporter construct. The HER2/ErbB2 promoter/reporter construct is preferably faithfully integrated and/or chromatinized, and/or capable of transcriptionally driving reporter gene expression.

One preferred HER2/ErbB2 promoter is a mutated HER2/ErbB2 promoter. A particularly preferred HER2/ErbB2 promoter contains up to 2 kb of sequence upstream of the TATAA-box directed +1 transcriptional start site, beginning at the SmaI restriction site ˜140 bp 5′ of the translation start site (ATG) and/or includes no more than 50 bp of the native HER2/ErbB2 5′ untranslated region (UTR). A particularly preferred promoter is an R06 human HER2/ErbB2 promoter construct

A preferred reporter gene encodes a transcript that has an in vivo half-life equal to or less than about 12 hours, more preferably equal to or less than about 6 hours. Certain preferred reporter genes include, but are not limited to β-galactosidase, chloramphenicol acetyl transferase (CAT), luciferase, fflux, green fluorescent protein, and red fluorescent protein.

In certain embodiments, the cell is a clonally selected human cell subline or a clonally selected non-human mammalian cell subline. Preferred cells include cells derived from a parental ErbB2-independent cell line (e.g. MCF-7, MDA-231, MDA-435, T47-D, etc.). Other particularly preferred cells include cells is derived from a parental ErbB2-dependent cell line (e.g. MDA-453, SKBr3, BT-474, MDA-463, SKOV3, MKN7, etc.). In certain embodiments, the cell is an ErbB2-independent cell that prior to integration of the promoter does not have an amplified HER2/ErbB2 promoter and its growth is not dependent on ErbB2 gene expression.

In certain embodiments, the cell used in the method comprises amplified copies of an endogenous HER2 or exogenous and stably introduced HER2/ERbB2 promoter and gene. In certain preferred embodiments, the test agent is a putative histone deacetylase (HDAC) inhibitor. A single test agent can be assayed, or the test agent can comprise a plurality of test agents. The contacting can be in any of a wide variety of formats (e.g. a microtiter (multi-well) plate). Particularly preferred formats are those suitable for high-throughput screening (e.g. in a high-throughput robotic device.). The method can additionally comprise entering a test agent that modulates (e.g. downregulate) activity of the HER2/ErbB2 promoter into a database of agents that modulate (e.g. downregulate) activity of a HER2/ErbB2 promoter.

In another embodiment, this invention provides a cell or cell subline useful for screening for an agent that modulates activity of a HER2/ErbB2 promoter. The cell or cell subline comprises a reporter gene operably linked to a faithfully integrated heterologous HER2/ErbB2 promoter, where the promoter is stably integrated into the genome of said cell. The cell or cell subline preferably comprises one or more of the promoter/reporter constructs described herein (e.g., a human HER2/ErbB2 promoter containing up to 2 kb of sequence upstream of the TATAA-box directed +1 transcriptional start site, beginning at the SmaI restriction site ˜140 bp 5′ of the translation start site (ATG) and including no more than 50 bp of the native HER2/ErbB2 5′ untranslated region (UTR)). The cell can be a human or a non-human mammalian cell or cell subline. Preferred cells include, but are not limited to those described herein.

In still another embodiment this invention provides a kit for screening for a modulator of HER2/ErbB2 promoter activity. The kit typically comprises a container containing a cell with a HER2 promoter/reporter construct as described herein. In certain embodiments, the container is a multi-well plate (e.g. a microtitre plate). The kit can further comprise instructional materials teaching the use of the cells in said kit for screening for modulators of HER2/ErbB2 activity. The instructional materials can additionally or alternatively describe the use of HDAC inhibitors to downregulate HER2/ErbB2 activity.

This invention also provides methods of downregulating an amplified or overexpressing promoter. The method comprises contacting a cell comprising the promoter with a histone deacetylase (HDAC) inhibitor. In preferred embodiments, the promoter comprises one or more DNaseI hypersensitivity (e.g., a promoter that regulates expression of a HER2/ErbB2/neu oncogene). In certain embodiments, the downregulating comprises silencing the expression of a gene or cDNA under control of the promoter. Preferred deacetylase (HDAC) inhibitors include, but are not limited to trapoxin B and trichostatin A, FR901228 (Depsipeptide), MS-275, sodium butyrate, sodium phenylbutyrate, Scriptaid, M232, MD85, SAHA, TAN-1746, HC-toxin, chlamydocin, WF-3161, Cly-2, and NSC #176328 (Ellipticine), and 6-(3-aminopropyl)-dihydrochloride) and NSC #321237 (Mercury,(4-aminophenyl)(6-thioguanosinato-N7,S6)-). In certain embodiments, the promoter is in a cancer cell (e.g., a breast cancer cell). In certain embodiments, the promoter is in a cell in a mammal (e.g. a human, or a non-human mammal).

This invention also provides a method of evaluating the responsiveness of a cancer cell to a histone deacetylase (HDAC) inhibitor. The method involves determining whether the cancer cell is a cell comprising amplified or overexpressed ERBB2, where a cell that comprises comprising amplified or overexpressed ERBB2 is expected to be more responsive to an HDAC inhibitor than a cell in which ERBB2 is at a normal level. In preferred embodiments, and average ErbB2 copy number greater than 1, more preferably greater than 1.5 and most preferably greater than 2 indicates that ERBB2 is amplified.

Also provided is a method of inhibiting the growth or proliferation of a cancer. The method involves determining whether said cancer comprises a cell comprising amplified or overexpressed ErbB2; and if the cancer comprises a cell comprising amplified or overexpressed ErbB2, contacting cells comprising the cancer with a histone deacetylase inhibitor. The contacting preferably comprises contacting the cancer cell with a deacetylase (HDAC) inhibitor in a concentration sufficient to downregulate or silence expression of a HER2/ErbB2/neu oncogene. Preferred histone deacetylase (HDAC) inhibitors include trapoxin B and trichostatin A, FR901228 (Depsipeptide), MS-275, sodium butyrate, sodium phenylbutyrate, Scriptaid, M232, MD85, SAHA, TAN-1746, HC-toxin, chlamydocin, WF-3161, Cly-2, NSC #176328 (Ellipticine), 6-(3-aminopropyl)-dihydrochloride, and NSC #321237 (Mercury,(4-aminophenyl)(6-thioguanosinato-N7,S6)-). In certain particularly preferred embodiments, the histone deacetylase (HDAC) inhibitor comprises a hydroxamic acid moiety. The HDAC inhibitor can be present in a pharmaceutically acceptable excipient.

In still yet another embodiment, this invention provides a kit for inhibiting the growth or proliferation of a cancer cell. Preferred kits comprise a histone deacetylase (HDAC) inhibitor; and instructional materials teaching the use of an HDAC inhibitor to downregulate expression of a HER2/ErbB2 oncogene. The HDAC inhibitor can be in a pharmaceutically acceptable excipient. Preferred HDAC inhibitors are in a unit dosage form.

This invention also provides a method of screening for an agent that downregulates expression of a HER2/ErbB2/neu oncogene. The method comprises contacting a cell comprising said a HER2/ErbB2/neu oncogene with a histone deacetylase; and detecting expression of a gene or cDNA under control of a HER2 promoter, where a decrease of expression of said gene or cDNA, as compared to a control, indicates that the agent downregulates expression of a HER2/ErbB2/neu oncogene. Preferred cells and/or promoters and/or reporters and/or promoter/reporter constructs include any of those described herein.

In another embodiment, this invention provides novel SNPs associated with the coding region of the ErbB2. proto-oncogene. The SNPs are indicators for altered risk, for developing ErbB2-positive cancer in a mammal. The SNPs identified herein can also be used for prognosis/prediction. The SNPs also provide novel prognostic/predictive tumor markers. The SNPs also provide new therapeutic targets.

Thus, in one embodiment, this invention provides a method of identifying an altered risk, for developing ErbB2-positive cancer in a mammal as compared to a healthy wild-type mammal. The method involves providing a biological sample from the mammal; and identifying the presence of a single nucleotide polymorphism selected from the group consisting of SNP-1, SNP-2, SNP-3, and SNP-4 as defined in Table 1, where the presence of the single nucleotide polymorphism indicates altered risk for developing ErbB2-positive cancer in said mammal as compared to a healthy wild-type mammal of the same species. In certain embodiments, the single nucleotide polymorphism indicates that said mammal has increased risk of developing ErbB2-positive cancer as compared to a healthy wild-type mammal of the same species. In certain embodiments, a homozygous occurrence of the SNP indicates greater risk than heterozygous occurrence of the SNP. The mammal can be a human, or a non-human mammal. In certain embodiments, the SNP is detected by detecting an SNP nucleic acid in the sample. The SNP nucleic acid can measured by hybridizing said nucleic acid to a probe that specifically hybridizes to an SNP nucleic acid (e.g. SNP-1, SNP-2, SNP-3, and/or SNP-4 or fragments thereof (e.g. fragment of at least 8 or 10 bp, preferably fragments of at least 12, 15, or 20 bp, more preferably fragments of at least 25, 30, or 40 bp, and most preferably fragments of at least 50 bp, or 100 bp.). The hybridization can be by any of number of convenient formats, e.g. a Northern blot, a Southern blot using DNA derived from the SNP RNA, an array hybridization, an affinity chromatography, and an in situ hybridization. The probe can be a member of a plurality of probes that forms an array of probes. In certain embodiments, the SNP nucleic acid is detected using a nucleic acid amplification reaction and/or a molecular beacon. The SNP can also be detected by detecting an SNP protein in the biological sample (e.g. via a method selected from the group consisting of capillary electrophoresis, a Western blot, mass spectroscopy, ELISA, immunochromatography, and immunohistochemistry).

This invention also provides a method of identifying increased risk for cancer progression and poor outcome in a mammal. The method involves providing a biological sample from said mammal; and identifying the presence of a single nucleotide polymorphism selected from the group consisting of SNP-1, SNP-2, SNP-3, and SNP-4 as defined in table 1, where the presence of one or more of these single nucleotide polymorphisms indicates increased risk for cancer progression and poor outcome in a compared to a wild-type mammal of the same species. In certain embodiments, homozygous occurrence of said SNP indicates greater risk than heterozygous occurrence of the SNP. The mammal can be a human or a non-human mammal (e.g. canine, equine, feline, porcine, etc.). The SNP can be detected by a variety of methods including, but not limited to any of the methods described herein.

Also provided is a method of subtyping a tumor. The method involves providing a biological sample comprising a cell from said cancer; and identifying the presence of a single nucleotide polymorphism selected from the group consisting of SNP-1, SNP-2, SNP-3, and SNP-4 as defined in table 1, where the presence of the single nucleotide polymorphism in the cell indicates a particular cancer subtype. In certain preferred embodiments, the cancer subtype is a subtype having enhanced oncogenic potential. Typically, homozygous occurrence of said SNP indicates greater risk than heterozygous occurrence of the SNP. The mammal can be a human or a non-human mammal. The SNP can be detected by a variety of methods including, but not limited to any of the methods described herein.

In still another embodiment, this invention provides a kit for detecting the presence of a single nucleotide polymorphism selected from the group consisting of SNP-1, SNP-2, SNP-3, and SNP-4 as defined in table 1. In certain embodiments, the kit comprises a container containing a probe that specifically hybridized under stringent conditions to a nucleic acid comprising a single nucleotide polymorphism selected from the group consisting of SNP-1, SNP-2, SNP-3, and SNP-4. The kit can optionally further comprise instructional materials teaching the detection of said single nucleotide polymorphism as an indicator of altered risk, for developing ErbB2-positive cancer in a mammal. In certain embodiments, the kit comprises a container containing an antibody that specifically binds to a polypeptide encoded by a nucleic acid comprising a single nucleotide polymorphism selected from the group consisting of SNP-1, SNP-2, SNP-3, and SNP-4. The kit can optionally further comprise instructional materials teaching the detection of the single nucleotide polymorphism as an indicator of altered risk, for developing ErbB2-positive cancer in a mammal.

In still another embodiment, this invention provides a nucleic acid that specifically hybridizes under stringent conditions to a nucleic acid comprising a single nucleotide polymorphism selected from the group consisting of SNP-1, SNP-2, SNP-3, and SNP-4. The nucleic acid can be a labeled nucleic acid.

### DEFINITIONS

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

An “amplified” promoter or promoter/reporter construct refers to a promoter or promoter/reporter construct that is present at an average copy number of at least 1/cell and is capable of overexpressing a reporter construct at a level exceeding that of the same cells bearing no reporter construct or bearing a control reporter construct not under the influence of the promoter sequence.

A “HER2/reporter construct” refers to the HER2 promoter (e.g. a mammalian, preferably a primate, and most preferably a human HER2 promoter) or fragment thereof operably linked to a reporter gene such that said HER2 promoter or promoter fragment regulates expression of said reporter gene.

The term “stably integrated” when used with respect to a HER2 promoter/reporter gene construct refers to the fact that both the HER2 promoter and the operably linked reporter gene/cDNA are stably integrated into the genome of the host cell. The construct is not substantially present as an episome or non-replicating but transcribing sequence transiently introduced into the cell's nucleus. In addition, sequence and linkage between the HER2 promoter and the reporter in the integrated construct are intact so that the reporter gene is not driven primarily by endogenous genomic sequence in proximity to the integrated construct.

The term “faithfully integrated”, when used in reference to a HER2/reporter construct indicates that the HER2/reporter construct is integrated into the genome of the host cell without recombination or other disruption of the construct's nucleotide sequence.

A “high ErbB2, ErbB2-positive, ErbB2-overexpressing cell or cell line” or an “ErbB2-dependent cell or cell line” refers to a cell or a cell line that typically overexpresses ErbB2 protein and mRNA in a constitutive manner, above that of normal or non-malignant cells, commonly, but not always, as a result of its underlying genomic/DNA amplification (e.g. an average ErbB2 gene copy number greater than 1.5, more preferably an average copy number greater than 2, still more preferably an average copy number greater than 3, or 4, or 5). Such cells or cell lines preferably include mammalian cells, more preferably primate cells, and most preferably human cells (e.g. MDA-453, SKBr3, BT-474, MDA-463, SKOV3, MKN7, etc.).

A “low ErbB2 cell or cell line” or an “ErbB2 independent cell or cell line” refers to a cell or cell line that typically does not overexpress ErbB2 and typically does not have an ErbB2 amplification (e.g. the average copy number is less than 1.5 and more typically about 1). Such cells or cell lines are preferably include mammalian cells, more preferably primate cells, and most preferably human cells (e.g. MCF-7, MDA-231, MDA-435, T47-D, etc.).

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Sanghui and Cook; Mesmaeker et al. (1994), Bioorganic & Medicinal Chem. Lett. 4: 395; Jeffs et al. (1994) J. Bimolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Sanghui and Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “operably linked” as used herein refers to linkage of a promoter to a nucleic acid sequence such that the promoter mediates/controls transcription of the nucleic acid sequence.

A “reporter gene” refers to gene or cDNA that expresses a product that is detectable by spectroscopic, photochemical, biochemical, enzymatic, immunochemical, electrical, optical or chemical means. Useful reporter genes in this regard include, but are not limited to fluorescent proteins (e.g. green fluorescent protein (GFP), red fluorescent protein (RFP), etc.) enzymes (e.g., luciferase, horse radish peroxidase, alkaline phosphatase β-galactosidase, chloramphenicol acetyl transferase (CAT), and others commonly used in an ELISA), and the like.

As used herein, the term “derived from a nucleic acid” (e.g., an mRNA) refers to a nucleic acid or protein nucleic acid for whose synthesis the referenced nucleic acid or a subsequence thereof has ultimately served as a template. Thus, a cDNA reverse transcribed or RT-PCR'd from an mRNA, an RNA transcribed from that cDNA, a DNA amplified from the cDNA, an RNA transcribed from the amplified DNA, etc., are all derived from the mRNA. In preferred embodiments, detection of such derived products is indicative of the presence and/or abundance of the original nucleic acid in a sample.

SNPs are single base pair positions in genomic DNA at which different sequence alternatives (alleles) in normal individuals in some population(s), wherein the least frequent allele has an abundance of 1% or greater. In practice, the term SNP is typically used more loosely than above. Single base variants in cDNAs (cSNPs) are usually classed as SNPs since most of these will reflect underlying genomic DNA variants. SNP datasets also typically contain variants of less than 1% allele frequency. The ‘some population’ component of the definition is limited by practical challenges of surveying representative global population samples.

An “SNP nucleic acid” refers to a nucleic acid comprising an SNP sequence.

The terms SNP polypeptide refers to a polypeptide encoded by an SNP nucleic acid.

The term “specifically binds”, as used herein, when referring to a biomolecule (e.g., protein, nucleic acid, antibody, etc.), refers to a binding reaction which is determinative of the presence of a biomolecule in a heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all, to other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part 1, chapt 2, Overview of principles of hybridization and the strategy of nucleic acid probe assays, Elsevier, NY (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions, e.g., containing formamide (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

### BRIEF DESCRIPTION OF THE DRAWINGS

FIG. 1 illustrates proximal promoter features regulating erbB2 transcription. Genomic landmarks an known positive-acting regulatory elements (EBS, NFY, R/N*, Sp1, AP2) are localized in relationship to the primary site of transcript initiation at +1 bp and a secondary site at −69 bp preferentially upregulated during promoter-driven erbB2 overexpression. Transactivator proteins though to bind these regulatory elements include Notch-activated RBPJκ (R/N*), and members of the Ets (EBS), Sp1 (Sp1, Ap2 (AP2) and CCAAT box binding protein (NFY) families. Other known regulatory features include the matrix attachment region (MAR) containing the 28 bp triplex-forming polypurine(GGA)-polypyrimidine(TCC) mirror repeat and an open-chromatin region of DNase-I hypersensitivity (HS) centered over the Ets binding site (EBS) and mirror-repeat element.

FIG. 2 illustrates the R06 construct. The box show to the right of BssHII is the most proximal region of the promoter containing putative tissue and development specific regulatory regions.

FIG. 3 illustrates a screening system for novel ErbB2 promoter-silencing drugs.

FIG. 4 illustrates intact ErbB2 promoter-reporter integration into MCF/R06pGL-9 genome.

FIG. 5 illustrates the DNase-I hypersensitivity site in endogenous ErbB2 promoter. The site is also present in chromatin-integrated but not transiently transfected R06pGL. DNA from nuclear preparations is Southern blotted and probed with promoter fragment (˜130 bp PstI-BssHII) designed to detect only the upstream endogenous ErbB2 hypersensitivity fragment.

FIG. 6 illustrates ErbB2 promoter-silencing candidates from identified from a high throughput screen.

FIGS. 7A through 7E illustrate results from a high throughput screen using a MCF/R06pGL-9. Results are 24 hours after administration of the test agent (drug). The upper lines () shows cell viability as determined using an MTT assay. The lower lines (▪) shows ErbB2 promoter function using a luciferase assay. FIG. 7A: TSA; FIG. 7B: NSC-131547 TSA; FIG. 7C: NSC-259968; FIG. 7D: NSC-176328; FIG. 7E: NSC-321237.

FIGS. 8A and 8B illustrate validation of ErbB2 promoter-targeted specificity: Trichostatin A (TSA) downregulates endogenous ErbB2 mRNA & protein. FIG. 8A shows Full-length (4.8 kb) ErbB2 transcript (see arrow) as expressed in ErbB2-amplified SKBr3 breast cancer cells and detected by Northern blotting total cell RNA. FIG. 8B shows full-length (185 kDa) ErbB2 protein (see arrow) as expressed in ErbB2-amplified MDA-453 breast cancer cells and detected by Western blotting total cell lysates.

FIG. 9 illustrates validation of NSC-176328 antitumor selectivity. ErbB2-positive human cancer cells are 8.5-fold more sensitive to NSC-176328 than a panel of ErbB2-negative cancer cells. In vitro antitumor activity against ErbB2-amplified BT-474 cells of free and liposome- or immunoliposome-encapsulated Ellipticine (NSC-176328). 6-3-aminopropylellipticine (AE; NSC 176328) was administered to cell cultures as free drug or after being encapsulated into liposomes (L-AE) or immunoliposomes (ILS-AE); the former prevent escape and cell exposure to free AE, the latter specifically internalize into and release AE within ErbB2 overexpressing tumor cells. The plot shows that free AE is cytotoxic to these ErbB2 overexpressing tumor cells with an LC₅₀ of 0.2 mg/ml; in contrast, against an NCI panel of 60 human cancer cell lines not overexpressing ErbB2 free AE showed an average LC₅₀ of 1.7 mg/ml. These data suggest that human cancer cell lines without ErbB2 amplification and overexpression are 8.5-fold more resistant to NSC-176328.

FIGS. 10A, 10B, and 10C illustrate the structure, genomic verification and trichostatin A (TSA) responsiveness of an ErbB2 promoter-reporter stably integrated into the MCF-7 subline, MCF/R06pGL-9. FIG. 10A. Restriction enzyme map of ErbB2-promoter-luciferase reporter construct (RO6pGL) above a map of known ErbB2 promoter features (EBS=Ets Binding Site, R/N*=RPBj/Notch, AP2, Sp1 and NFY response elements) including transcription initiation sites (Inr −69 and Inr +1) and DNase I hypersensitivity (HS) site, contained within the 500 bp Sma1-Sma1 promoter fragment as has been previously detailed (10). FIG. 10B: Southern analysis of the MCF/RO6pGL-9 subline demonstrating restriction fragment lengths consistent with faithful genomic integration of the ErbB2 promoter-reporter. Hybridization probe was a 257 bp Pst1-Sma1 ErbB2 promoter fragment. FIG. 10C: High-throughput screening (HTS) assay for luciferase activity and cell viability (MTT assay) from 96-well replicate cultures of MCF/RO6pGL-9 cells showing their responsiveness to a 24 hour treatment with the indicated TSA doses.

FIGS. 11A and 11B show trichostatin A (TSA) responsiveness and DNase I hypersensitivity comparisons between the genomically integrated vs. episomally introduced ErbB2 promoter-reporter construct, RO6pGL, in MCF-7 cells. FIG. 11A: Luciferase activity (relative luminometer units) of the MCF/RO6pGL-9 subline compared to parental MCF-7 cells transiently transfected with the same ErbB2 promoter-reporter plasmid (RO6pGL) and after 24 hour treatment with 0.4 μM TSA, normalized against their respective non-treatment (vehicle only) control conditions. FIG. 11B: DNase 1 hypersensitivity analysis (described in Methods) of nuclei from MCF/RO6pGL-9 vs. MCF-7 cells transiently transfected with RO6pGL, following similar culture treatments with TSA.

FIGS. 12A and 12B show a comparison of TSA effects on the transcript expression and DNase 1 hypersensitivity of ErbB2 and ESX genes. FIG. 12A: DNase 1 hypersensitivity analysis of nuclei from SKBR3 cells following treatment with (+) or without (−) 24 hour exposure to 0.4 μM TSA. Southern blot prepared from Hind III digested DNA was probed first with an ErbB2 promoter probe followed by an ESX cDNA probe for correct hypersensitive fragment band assignments. Lanes without DNase 1 treatment clearly define the endogenous 2.5 kb ErbB2 HindIII fragment and 11 kb ESX HindIII fragment. FIG. 12B: Northern blot of total RNA isolated from SKBR3 cells following 24 hour culture treatment with (+) or without (−) 0.4 μM TSA, probed first with ErbB2 cDNA (left panel) and then reprobed with ESX cDNA (right panel) to reveal their respective (4.8 kb, 2.2 kb) transcript bands.

FIGS. 13A and 13B show the influence of TSA on ErbB2 and ESX transcript synthesis and stability. FIG. 13A: Nucler run-offs showing radiolabelled and newly synthesized RNA from nuclei of SKBR3 cells pretreated (+) or not pretreated (−) for 5 hours with 0.4 μM TSA, hybridized to membranes slotted with ErbB2 carboxy-terminus and ESX cDNA fragments. FIG. 13B: Northern blot of total RNA from SKBR3 cells similarly treated for 5 hours +/−0.4 μM TSA or +/−10 μg/ml of the RNA polymerase inhibitor, actinomycin D (Act D), probed first with ErbB2 cDNA (left panel) and then with ESX cDNA (right panel) to reveal the treatment effects on their total intracellular transcript (4.8 kb, 2.2 kb) levels.

FIG. 14 shows ErbB2 protein levels following TSA treatment of various ErbB2 overexpressing breast cancer cell lines. Western blots of whole cell extracts from four different cell lines (SKBR3, MDA-453, BT-474, MCF/HER2-18) following treatment with 0.4 μM TSA for the indicated times (hours). Membranes were probed with antibodies to the ErbB2 and α-tubulin proteins, with the resulting band intensities as indicated. SKBR3, MDA-453, and BT-474 cells overexpress 185 kDa ErbB2 protein from their endogenously amplified oncogenes, while MCF/HER2-18 cells overexpress 185 kDa ErbB2 protein from a genomically integrated but ectopically introduced ErbB2 expression vector lacking native ErbB2 promoter and non-coding cDNA sequences.

### DETAILED DESCRIPTION

This invention pertains to the development of a screening system to identify (screen for) HER2 promoter silencing agents. Such agents are expected to be of therapeutic value in the treatment of cancers characterized by HER2 amplification/upregulation. In addition, this invention pertains to the discovery that histone deacetylase (HDAC) inhibitors like sodium butyrate and trichostatin A (TSA), in a time and dose dependent fashion can silence genomically integrated and/or amplified/overexpressing promoters, such as that driving the HER2/ErbB2/neu oncogene, resulting in inhibition of endogenous and/or exogeneous gene products including transcripts and protein, and the subsequent induction of tumor/cell growth inhibition, apoptosis and/or differentiation. Moreover, it was a discovery of this invention that such agents are likely to be of greater efficacy in cancer cells and cancers characterized by HER2-dependence, HER2-overexpression and/or HER2 amplifications.

### The Screening System.

In one embodiment, this invention provides a novel screening system to screen for agents that modulate (e.g. upregulate or downregulate) activity of the HER2 promoter. In general, the screening system comprises a cell comprising a reporter gene operably linked to a heterologous HER2/ErbB2 promoter, where the promoter and the reporter are stably integrated into the genome of the cell. In this system, the reporter system is one in which HER2 promoter activity reflects endogenous chromatin-regulated promoter control. The reporter is preferably one that provides a high amplitude, short half-life signal that is rapidly and conveniently measurable in response to complete or partial promoter repression.

The cell is preferably a clonally selected subline. It was a discovery of this invention that clonally selected sublines can be characterized as HER2-independent and low expressing (e.g. from the parental lines MCF-7, MDA-231, MDA-435, T47-D, etc.) or HER2-dependent and high expressing (e.g. from the parental lines MDA-453, SKBr3, BT-474, MDA-463, SKOV3, MKN7, etc.) and that whether the parental line is HER2-dependent or HER2-independent dramatically effects the cells ability to accept and genomically integrate a heterologous HER2 promoter/reporter construct and/or grow in a clonal fashion to form a subline containing the stably and faithfully integrated HER2 promoter/reporter construct.

In particular, it was observed that it was initially difficult to obtain stable sublines bearing the integrated and expressing HER2 promoter/reporter construct from parental HER2-dependent cells Without being bound to a particular theory, it is believed that these HER2-dependent cells are representative of HER2-dependent human cancers and loose their ability to overexpress their essential endogenous HER2 growth factor receptor when an exogenous HER2 promoter/reporter construct is genomically introduced and sequesters or steals essential transcription factors or co-factors from the endogenous HER2 promoter and oncogene, We believe that this promoter-stealing mechanism may also form the basis of new promoter-silencing therapeutics.

Creation of stably integrated HER2 promoter/reporter constructs in these HER-dependent cell systems can be facilitated by titrating down the HER2 promoter/reporter copies and/or less transcriptionally active promoter/reporter constructs with reduced stealing of factors/co-factors from the endogenous HER2 oncogene's promoter.

While both the low endogenous erbB2 and high endogenous erbB2 reporter systems (cells) can be used to screen for modulators of HER2 driven transcription, the low erbB2 cell sublines are particularly well suited for such screening systems. In the high erbB2 cells, erbB2 expression is essential for cell survival. Down regulation of HER2 promoter activity by a test agent can therefore result in death of the cell. Without subsequent assays, one cannot tell if the test agent worked though its activity on HER2 expression or by killing the cell through some other mechanism.

In contrast, the low endogenous ErbB2 cells do not require ErbB2 activity for survival. Inhibition of HER2 promoter activity will not kill or retard growth of the cell. Thus it is possible to directly assay test agents for their ability to alter (e.g. down-regulate) HER2 promoter activity without necessarily affecting cell growth and thus without inducing indirect affects on the reporter assay system as a result of less specific or more general influences on cell growth and metabolism.

Stable integration and expression of exogenous genes into parental cell lines that are subcloned to produce stably transfected sublines can be made according to standard methods, well known to those of skill in the art (see, e.g., Liu et al. (1989) Oncogene 4: 979-984; Benz et al. (1992) Breast Cancer Res. Treat. 24: 85-95; Scott et al. (1993) Mol. Cell. Biol. 13: 2247-2257)

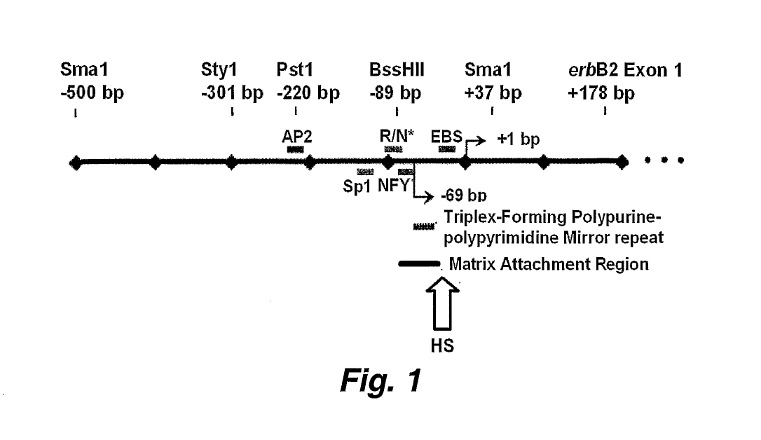

Constructs comprising a HER2 promoter operably linked to a nucleic acid encoding a reporter can be made using standard cloning methods well known to those of skill in the art. The structure of the HER2 promoter is well characterized (see, e.g., Scott et al. (1994) J. Biol. Chem. 269: 19848-19858; and Scott et al. (2000) Oncogene, 19: 6490-6502). The most critical regulatory elements are located within the promoter's proximal 200 bp (relative to the major transcriptional start sites at +1 and −69) (Ishii et al. (1987) Proc. Natl. Acad. Sci., USA, 84(13): 4374-4378; Hudson et al. (1990) J. Biol. Chem. 265(8): 4389-4393; Mizuguchi et al. 91994) FEBS Lett., 348(1): 80-88; Chen and Gill (1994) Oncogene, 9(8): 2269-2276; Grooteclaes et al. (1994) Cancer Res., 54(15): 4193-4199; Scott et al. 91994) J. Biol. Chem. 269931): 19848-19858; Bosher et al. (1995) Proc. Natl. Acad. Sci., USA, 92(3): 744-747; Chen et al. (1997) J. Biol. Chem. 272(22): 14110-14114; Raziuddin et al. (1997) J. Biol. Chem. 272(25): 15715-15720). This region contains binding sites for ubiquitous transcription factors AP-2, Sp1, NF-Y, Elf-1 (Ishii et al. (1987) Proc. Natl. Acad. Sci., USA, 84(13): 4374-4378; Bosher et al. (1995) Proc. Natl. Acad. Sci., USA, 92(3): 744-747) and tissue development-specific transcription factors Notch-activated RBPJκ (R/N) and ESX (Chen et al. (1997) J. Biol. Chem. 272(22): 14110-14114; Chang et al. (1997) Oncogene, 14913): 1617-1622) as shown in FIG. 1. In certain preferred embodiments the promoter construct comprises the R06 human HER2/ErbB2 promoter construct (see, e.g., Scott et al. (1994) J. Biol. Chem. 31: 19848-19858).

We have identified a single region of open chromatin associated with localized hypersensitivity to endonucleases (DNAase I, S1): this particular unique hypersensitivity site is located over a GAA mirror repeat sequence just upstream of the essential ETS binding site (EBS) at −35 bp (Chang et al. (1997) Oncogene, 14913): 1617-1622). Without being bound to a particular theory it is believed that this region can also exist in an endogenous triple helix configuration known as H-DNA.

Without being bound to a particular theory, we believe that constitutive (overexpressing) states of gene expression are specified by distinctly activated promoter confirmations. We believe that in HER2 overexpressing cells the activated HER2 promoter confirmation is associated with a more intense hypersensitivity site over the GAA mirror repeat and matrix-binding region, is bound by at least one member of the Ets transcription factor family as well as the notch-activated binding protein, RBPJ^{κ}, facilitating rapid and preferential transcript re-initiation at −69 bp in addition to initiation at +1 bp. In low HER2 expressing cells, a different combination of transcription factors and cofactors bind to this same promoter region in association with H-DNA stabilization, a promoter DNA structure associated with retardation of both gene transcription and replication, and resulting in low or basal levels of transcript production virtually all of which are initiated at +1 bp.

The construct is designed to provide an exogenous HER2 promoter-reporter construct that can be stably integrated into a endogenous chromatin environment. This system provides a more accurate determination of promoter function than that provided using transient transfection protocols in which a transfected reporter construct is transiently and episomally active, without chromatin, nucleosomes, and/or nuclear matrix association, and without assuming a higher order promoter architecture or without immediate exposure to such transcription factors and cofactors that typically upregulate HER2 oncogene expression.

In preferred embodiments, the constructs comprise at least 0.1 kb, preferably at least 0.125 kb, more preferably at least 0.2 kb, and most preferably at least 0.5 kb 1 kb, or 2 kb of proximal promoter sequence driving (operably linked to) a reporter gene. Suitable reporter genes are well known to those of skill in the art. Such reporter genes include, but are not limited to (luciferase, green fluorescent protein, beta galactosidase, chloramphenicol acetyl transferase, and the like). Particularly preferred reporter genes provide a high amplitude and short half-life protein signal that is rapidly (e.g. within 48 hr) and conveniently measurable in response to complete or partial promoter repression in contrast to the endogenous gene response (HER2 transcript and protein expression). Given the approximately 24 hr half-life of endogenous HER2 transcripts and comparable half-life for the receptor protein, full repression of the endogenous promoter for 72 hours would not be expected to decrease HER2 protein levels more than 50%. In contrast, given the 6 and 3 hr half-lives respectively, e.g. of luciferase transcript and protein, reporter gene expression should be minimal within 72 hours of promoter repression. Particularly preferred promoters have a half-life of less than about 12, hours, more preferably less than about 6 hours.

The HER2 promoter-reporter gene constructs (e.g., 2.0, 0.5, 0.125 kb of proximal promoter sequence driving a reporter gene) are stably transfected into cell lines (e.g. breast epithelial cell lies), e.g. by standard methods known to those of skill in the art.

In certain embodiments, rather than using expression vectors that contain viral origins of replication, host cells can be co-transformed with the HER2 promoter/reporter constructs described above, and a selectable marker. Following the introduction of the foreign DNA by lipid-based or other methods (e.g. calcium phosphate precipitation), transfected cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that, in turn, can be clonally selected and expanded into cell sublines. This method may advantageously be used to engineer cell lines that express the stably integrated reporter construct.

A number of selection systems can be used, including but not limited to the herpes simplex virus thymidine kinase (Wigler et al. (1977) Cell 11:223), hypoxanthine-guanine phosphoribosyltransferase (Szybalska and Szybalski (1962) Proc. Natl. Acad. Sci., USA, 48: 2026), and adenine phosphoribosyltransferase (Lowy et al. (1980) Cell 22: 817) genes can be employed in tk, hgprt⁻, or aprt⁻ cells, respectively. Also, antimetabolite resistance can be used as the basis of selection for the following genes: dhfr, which confers resistance to methotrexate (Wigler et al. (1980) Proc. Natl. Acad. Sci., USA, 77:3567; O'Hare et al. (1981) Proc. Natl. Acad. Sci., USA, 78:1527); gpt, which confers resistance to mycophenolic acid (Mulligan and Berg (1981) Proc. Natl. Acad. Sci., USA, 78:2072); neo, which confers resistance to the aminoglycoside G-418 (Colberre-Garapin et al. (1981) J. Mol. Biol. 150:1); and hygro, which confers resistance to hygromycin (Santerre et al. (1984) Gene 30:147).

Any technique known in the art may be used to introduce the HER2/reporter construct into cells. Such techniques include, but are not limited to microinjection (U.S. Pat. No. 4,873,191); retrovirus mediated gene transfer (see, e.g., Van der Putten et al. (1985) Proc. Natl. Acad. Sci., USA, 82:6148-6152); gene targeting; electroporation, calcium phosphate precipitation (Liu et al. (1989) Oncogene 4: 979-984; Benz et al. (1992) Breast Cancer Res. Treat. 24: 85-95; Scott et al. (1993) Mol. Cell. Biol. 13: 2247-2257, 1993), and the use of transfection reagents (e.g., Lipofectamine™ (Gibco-BRL), effectene (Qiagen), fugene (Roche), and the like).

In certain preferred embodiments, transfection reagents or retroviral methods are used to introduce the HER2/reporter construct. Preferred retroviral methods employ a pBabe or MFG-based vector in combination with the Phoenix transient retroviral packaging system (Grignani et al. (1998) Cancer Res., 58(1): 14-19) for retroviral infection and genomic integration of the HER2 reporter constructs. Preferred non-retroviral transfection methods employ the lipid-based reagents lipofectamine, effecten, or fugene.

In particularly preferred embodiments, stable transfected monoclonal sublines are selected on the basis of antibiotic resistance by virtue of co-transfection of a marker gene (e.g., neomycin phosphotransferase) and selective growth on serial passaging over many weeks in the presence of 0.5 mg/ml G418 antibiotic. The presence, integrity, and copy number of stably integrated DNA in antibiotic-resistant clones is determined by PCR and/or Southern blotting.

Examples of these techniques and instructions sufficient to direct persons of skill through the cloning exercises described above are found in Berger and Kimmel, Guide to Molecular Cloning Techniques, Methods in Enzymology, volume 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement).

Reporter gene activity in cell lysates is analyzed by standard methods (e.g., luminometry for luciferase activity). Monoclonal and polyclonal cell sublines are established and characterized. Polyclonal populations are used to determine promoter function in low versus high HER2 expressing cells. Basal reporter activity and upregulation in response to known stimulating agents (e.g. camp, TPA, cell density) are assayed (Hudson et al. (1990) J. Biol. Chem. 265(8): 4389-4393; Taverna et al. (1994) Internat. J. Cancer, 56(4): 522-528). Transactivation experiments can also be carried out in low and high HER2 expressing integrants to verify the upregulating or downregulating activity of specific transcription factors (e.g. Elf-1, notch-activated R/N, etc.).

In preferred embodiments, monoclonal sublines are selected for the testing/screening of agents that modulate (e.g. downregulate) HER2 promoter activity.

### Running the Assay.

The assays of this invention typically contacting a cell comprising the stably integrated HER2/reporter construct described above with a test agent; and detecting expression of the reporter gene where a change in expression of the reporter gene, e.g., as compared to a control indicates that the test agent modulates activity of the HER2/ErbB2 promoter. Preferred test agents will downregulate or fully silence the HER2 promoter.

The “test agent” can be virtually any chemical compound. It can exist as a single isolated compound or can be a member of a chemical (e.g. combinatorial) library. In a particularly preferred embodiment, the test agent will be a small organic molecule. In certain embodiments, the test agent is a known, putative, or potential HDAC inhibitor. In certain particularly preferred embodiments, the test agent comprises a hydroxamic acid moiety.

Where the change in expression of the reporter gene is determined with respect to a control, the control can be a negative control (e.g. the same assay absent the test agent or with the test agent at a lower concentration). Alternatively, or in addition, the control can be a positive control (e.g. the same assay run with a agent known to induce transcription under the control of the HER2 promoter).

A change in expression of the reporter gene includes any detectable change in expression of the reporter gene. In preferred embodiments, the change is a statistically significant change, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the change is at least a 10% change, preferably at least a 20% change, more preferably at least a 50% change and most preferably at least a 90% change.

The screening methods of this invention can take place in a wide variety of formats. Thus, for example a single test agent can be screened with one or more cell lines. In addition, multiple agents can be screened against one or more cell lines at the same time. This can be accomplished by contacting different test agents with each cell line in a separate reaction vessel or well. Alternatively, multiple test agents can be assayed in a single assay. Those assays that test positive are then deconvolved in subsequent assays to determine which of the test agents in the positive screen was responsible for the positive signal.

The assays can be run in any convenient format. In particularly preferred embodiments, the assays are run in a multi-well format (e.g. 96 well plate, 384 well plate, etc.) suitable for high throughput screening.

High Throughput Screening for Agents that Modulate HER2 Regulated Gene Expression.

As indicated above, the assays of this invention are also amenable to “high-throughput” modalities. Conventionally, new chemical entities with useful properties (e.g., downregulation of HER2) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

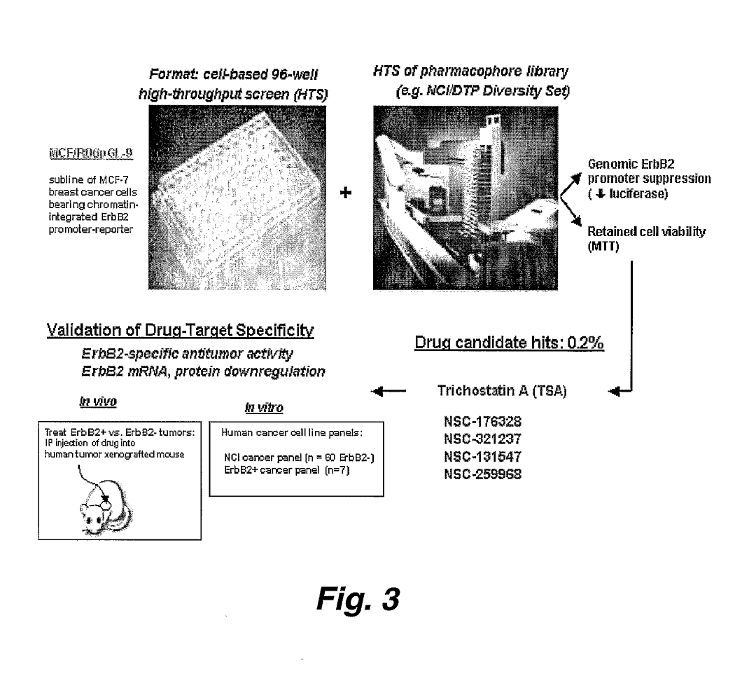

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics. One high throughput screening approach is illustrated in FIG. 3.

Combinatorial Chemical Libraries for Modulators of HER2 Promoter Activity.

The likelihood of an assay identifying a modulator of HER2 promoter activity is increased when the number and types of test agents used in the screening system is increased. Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

High Throughput Assays of Chemical Libraries for Agents that Modulate HER2 Promoter Activity.

Any of the assays for agents that modulate HER2 promoter activity are amenable to high throughput screening. As described preferred assays detect inhibition of expression of a reporter gene (e.g. luciferase) by the test compound(s). High throughput assays for the presence, absence, or quantification of particular reporter gene products are well known to those of skill in the art.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### HDAC Inhibitors to Down-Regulate HER2 Driven Expression.

In another embodiment this invention pertains to the discovery that histone deacetylase (HDAC) inhibitors like sodium butyrate and trichostatin A (TSA), can silence genomically integrated and/or amplified/overexpressing promoters such as that driving the HER2/ErbB2/neu oncogene in a time and dose dependent fashion. This results in inhibition of gene products including transcripts and protein, and subsequent tumor/cell growth inhibition, apoptosis and/or differentiation.

Histone deacetylase inhibitors (HDAC-I) like butyrate, the depsipeptide FK228, the fungal metabolite and antiprotozoal apicidin, the synthetic benzamide derivatives MS-275 and CI-994 (Pfizer), and the hydroxamic acid derivatives trichostatin A (TSA) and suberoylanilide hydroxamic acid (SAHA; MSKCC) all have known antiproliferative effects against breast, lung, prostate and other cancer cells as well as tumors in mice, due to their acetylation of histone (H3, H4) and non-histone proteins resulting in the transcriptional induction of p21Waf1, p16INK4A, and other cell cycle arresting factors. leading to terminal cytodifferentiation, senescence and/or tumor cell apoptosis (Marks et al. (2000) J. Natl. Cancer Inst. 92: 1210-1216; Weidle and Grossmann (2000) Anticancer Res. 20: 1471-1485; Yoshida et al. (1990) J. Biol. Chem. 265: 17171-17179; Finnin et al. (1999) Nature 401: 188-193; Darkin-Rattray (1996) Proc. Natl. Acad. Sci., USA, 93: 13141-13147; Saito et al. (1999) Proc. Natl. Acad. Sci., USA, 96: 4592-4597; Butler et al. (2000) Cancer Res. 60: 5165-5170) Why these agents are selective for cancer cells and the molecular basis for their antitumor selectivity remain unknown.

TSA, administered parenterally (ip, sc) at histone-acetylating doses (up to 5 mg/kg) to rats and mice, has shown potent antitumor activity against a carcinogen-induced mammary cancer and exhibits no ill effects to either adult or embryonic mice (Vigushin et al. (1999) Clin. Cancer Res. 5 (suppl.): #239 (abstract, Proc. AACR-NCI-EORTC Int. Conf.); Nervi et al. (2001) Cancer Res. 61: 1247-1249). The newer HDAC-Is, orally active CI-994 and iv administered SAHA, are also well tolerated, have entered Phase-II clinical testing and are showing antitumor activity against various refractory human tumors (Kimmel et al. (2001) Proc. Amer. Soc. Clin. Oncol. 20: 87a; Kelly et al. (2001) Proc. Amer. Soc. Clin. Oncol. 20: 87a). Microarray studies indicate that of the ˜7% of genes whose cellular expression are affected by an HDAC-I (i.e. butyrate, TSA), most (6×) are upregulated while very few are downregulated within 48 h of cell treatment (Mariadason et al. (2001) Cancer Res. 60: 4561-4572).

In contrast, using the assays of this invention to screen for ErbB2 promoter-silencing agents, we found that both sodium butyrate and TSA (at a dose that retains cell viability for >48 h) significantly represses a breast cancer genomically integrated ErbB2 promoter-reporter within 24 h of treatment. In addition, this same TSA dose selectively and more substantially reduces endogeneous ErbB2 transcript and protein levels in ErbB2-positive MDA-453 and SkBr3 cell lines.

Without being bound by a particular theory, HDAC inhibitor's ability to silence such promoters may work either by directly altering the promoter's chromatin structure (e.g. localized histone acetylation) or by modifying acetylated non-histone proteins that bind to and regulate transcription off that promoter (e.g. Ets factors or components of the basal transcription machinery). This therapeutic promoter repressing mechanism is also paradoxical to the stimulatory response observed with these same HDAC inhibitors on gene expression constructs introduced transiently or on other endogenously integrated and chromatinized promoters such as the promoter for the acetylated Ets factor, ESX. It also appears to be more selective for certain gene transcripts.

In general, we believe that ErbB2-positive tumors are particularly sensitive targets for HDAC-I therapy. Thus, in certain embodiments, this invention contemplates a method of evaluating the responsiveness of a tumor to treatment with a histone deacetylase inhibitor (HDAC). The method involves assaying the cancer cell(s) for erbB2 copy number and/or expression level. Elevated erbB2 copy number and/or expression level indicates that the cancer cell(s) are erbB2-dependent cells and thus will show greater sensitivity (responsiveness) to HDACs, particularly to HDACs comprising a hydroxamic acid moiety.

Histone deacetylase inhibitors are well known to those of skill in the art. Examples of known histone deacetylase inhibitors include, but are not limited to butyric acid, MS-27-275, SAHA, Trichostatin A, Oxamflatin, Depsipeptide, Depudecin, Trapoxin, HC-toxin, chlamydocin, Cly-2, WF-3161, Tan-1746, apicidin and analogs thereof (see, e.g., Marks et al. (2000) J. Nat. Cancer Inst., 92(15): 1210-1216). In particular it is noted that HC-Toxin is described in Liesch et al. (1982) Tetrahedron 38: 45-48; Trapoxin A is described in Itazaki et al. (1990) J. Antibiot. 43: 1524-1532; WF-3161 is described in Umehana et al. (1983) J. Antibiot. 36: 478-483; Cly-2 is described in Hirota et al (1973) Agri. Biol. Chem. 37: 955-56; chlamydocin is described in Closse et al. (1974) Helv. Chim. Acta 57: 533-545 and Tan 1746 is described in Japanese Patent No. 7196686 to Takeda Yakuhin Kogyo K K. Particularly preferred HDACs include HDACs comprising a hydroxamic acid moiety or a derivative thereof (e.g. hydroxamic acid-based hybrid polar compounds (HPCs)).

The HDACs can be formulated and administered according to standard methods well known to those of skill in the art (see, e.g., references cited above). Various HDACs can be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The HDACs and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The HDACs and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well-known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a disease (e.g., cancer and/or associated conditions) in an amount sufficient to cure or at least partially arrest the disease and/or its symptoms (e.g. to reduce cancer cell growth and/or proliferation) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the HDACs are administered orally (e.g. via a tablet) or as an injectable in accordance with standard methods well known to those of skill in the art. In other preferred embodiments, the HDACs may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

### Kits.

This invention also provides kits for practice of the methods described herein. Preferred kits comprise a container containing a cell comprising a stably-integrated HER2 promoter/reporter construct as described herein. Such kits can optionally include various reagents for use as controls, buffer solutions, reagents for detecting reporter gene products and so forth.

In addition, the kits can, optionally, include instructional materials containing directions (i.e., protocols) for the practice of the methods of this invention. Preferred instructional materials provide protocols utilizing the kit contents for screening for agents that downregulate HER2 promoter driven gene expression or for detecting erbB2 levels in cells (e.g. cancer cells) and/or for administering HDACs for inhibiting HER2 promoter driven gene transcription e.g., in a cancer cell. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Single Nucleotide Polymorphisms (Snps) within the Erbb2 Proto-Oncogene

In another embodiment, this invention pertains to the identification of a number of single nucleotide polymorphisms (SNPs) within the ErbB2 proto-oncogene. In particular, we searched across >140 kb of ErbB2 genomic database sequence and identified four coding region SNPs (see, Table 1). These SNPs lie within codons for amino acids 654 and 655 in the transmembrane domain, 927 in the tyrosine kinase domain, and 1170 in the intracellular regulatory domain (ICRD).

TABLE 1
SNPs associated with the ErbB2 proto-oncogene.

| | Description/location of SNP | |
|---|---|---|
| SNP | within ErbB2 proto-oncogene* | Database No. |
| SNP-1 | Ala1170Pro substitutes a C for a G position | rs1058808 |
| | at cDNA position 3658 resulting in the amino | Genbank No: |
| | acid substitution of proline (Pro) for alanine | Hs. 173664 M |
| | (Ala) at amino acid position 1170 (intracellular | 11730 |
| | regulatory domain (ICRD). | |
| SNP-2 | Amino acid 654 within transmembrane domain. | |
| SNP-3 | Val/655/Ile substitutes Ile for Val at amino acid | |
| | 655 within the transmembrane domain. | |
| SNP-4 | Amino acid 927 in the tyrosine kinase domain. | |

*Nucleotide and amino acid numbers as oriented by Coussens et al. (1985) Science, 230: 1132-1139

One of the transmembrane domain SNPs has formerly been reported (Val/655/Ile) and was linked to increased breast cancer susceptibility but this was controversial and evidence for this SNP in human cancers has never been presented. Using a bank of normal and breast cancer DNA samples blindly genotyped by SnaPshot PCR (Applied Biosystems), we focused on the putative 927 and 1170 SNPs; we could not confirm the presence of the 927 SNP in any of these samples, but found evidence for both Ala (wildtype) and Pro 1170 variants occurring with an overall 64% Ala allele frequency and a 36% Pro allele frequency and no significant difference between normal (n=8) and breast tumor (n=58) samples in these allele frequencies. ErbB2 protein, mRNA and gene copy number assays were used to subdivide the breast tumors into ErbB2-positive (n=11) and ErbB2-negative (n=47) subgroups. While 19% of all tumors possessed the homozygous Pro variant, this genotype was five-fold more frequent in the ErbB2-positive tumors as compared to the ErbB2-negative tumors (55% vs. 11%), and these tumor subgroups showed highly significant (p=0.004) frequency differences across all three (Ala/Ala, Ala/Pro, Pro/Pro) genotypes.

Having determined the association of the SNPs identified herein with the ErbB2 proto-oncogene, these SNPs lend themselves to a large number of applications. For example, the SNPs can be used in risk assessment. This involves the genotyping of normal cells (e.g., blood, epithelial, other cells) which can demonstrate increased risk for developing ErbB2-positive cancer, for example, if the PRO-encoding allele is present, especially if the organism tested is homozygous for the Pro-encoding allele, and relatively less risk if the organism is homozygous for Ala encoding allele.

The SNPs identified herein can also be used for prognosis/prediction. In this instance, genotyping cancer cells (e.g. ErbB2+ subtype) can demonstrate increased risk for cancer progression and poor patient outcome despite standard therapy, e.g., if the Pro encoding allele is present, especially if the organism is homozygous for the Pro encoding allele, and relatively less risk if the organism is homozygous for Ala encoding allele.

The SNPs also provide novel prognostic/predictive tumor markers. Rapid DNA, RNA, or protein based methods of detecting the Pro encoding allele or its gene product is possible through a number of commercial assay kits enabling distinction of the Pro1170 amino acid or its encoding sequence from the wildtype Ala1170 amino acid or its encoding sequence and subtyping of ErbB2+ cancers. In particular, despite overall reduced frequency of individuals with homozygous Pro genotype (Pro/Pro), heterozygotic individuals (normal cell genotype of Ala/Pro het) can develop homozygous (Pro/Pro) ErbB2+ tumors due to selective gene amplification of this allele. The selective pressures allowing for outgrowth of Pro/Pro genotype ErbB2+ cancers could be multiple: e.g. enhanced oncogenic potential of Pro encoding ErbB2 receptor tyrosine kinase, altered tumorigenic signal pathways associated with this ErbB2 variant, and/or altered immunogenicity or immune surveillance response to Pro encoding ErbB2.

The SNPs also provide new therapeutic targets. Immunotherapies or drugs capable of specifically targeting the Pro1170 variant of ErbB2 from the wildtype Ala1170 variant of ErbB2 are readily created.

### Detection of SNPS.

Using the information provided herein, the SNPs described herein, can readily be detected in a biological sample. Methods of detecting SNPs are well known to those of skill in the art (see, e.g., U.S. Pat. No. 6,322,980, SnaPshot PCR (Applied Biosystems), and the like). In general the methods involve either detecting the genomic DNA encoding the SNP, the mRNA encoding the SNP, and/or the SNP protein.

A) Nucleic-Acid Based Assays.

1) Target Molecules.

The SNPs of this invention can be detected by detecting SNP DNAs and/or SNP RNAs. In order to detect the SNP nucleic acid expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., genomic DNA, mRNA, nucleic acid derived from mRNA, etc.) is, in certain preferred embodiments, isolated from the sample according to any of a number of methods well known to those of skill in the art. Methods of isolating DNA and RNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part 1. Theory and Nucleic Acid Preparation, Elsevier, N.Y. and Tijssen ed.

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA_{—}86: 1173), self-sustained sequence replication (Guatelli et al. (1990) Proc. Nat. Acad. Sci. USA 87: 1874), dot PCR, and linker adapter PCR, etc.).

2) Hybridization-Based Assays.

Using the SNP sequences provided detecting and/or quantifying the SNPs can be routinely accomplished using nucleic acid hybridization techniques (see, e.g., Sambrook et al. supra). For example, one method for evaluating the presence, absence, or quantity of SNP reverse-transcribed cDNA involves a “Southern Blot”. In a Southern Blot, the DNA (e.g., reverse-transcribed SNP mRNA), typically fragmented and separated on an electrophoretic gel, is hybridized to a probe specific for the SNP. Comparison of the intensity of the hybridization signal from the SNP probe with a “control” probe (e.g. a probe for a “housekeeping gene) provides an estimate of the relative expression level of the target nucleic acid.

Alternatively, the SNP mRNA can be directly detected/quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target SNP mRNA. Appropriate controls (e.g. probes to housekeeping genes) provide a reference for evaluating relative expression level.

An alternative means for detecting the SNP is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to detect/measure the SNP. In such amplification-based assays, the target nucleic acid sequences (i.e., SNP-1) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., SNP) in the original sample. Comparison to appropriate (e.g. healthy tissue or cells unexposed to the test agent) controls provides a measure of the SNP transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for SNP1 are sufficient to enable one of skill to routinely select primers to amplify any portion of the gene.

4) Hybridization Formats and Optimization of Hybridization Conditions.

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemiluminescent agents, enzymes, and antibodies that can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization. With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of SNPs can be full length or less than the full length of the SNP. Shorter probes are empirically tested for specificity. Preferred probes are sufficiently long so as to specifically hybridize with the SNP target nucleic acid(s) under stringent conditions. The preferred size range is from about 20 bases to the length of the SNP coding region, more preferably from about 30 bases to the length of the SNP mRNA, and most preferably from about 40 bases to the length of the SNP mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of SNP nucleic acids, SNPs can be detected and/or quantified by detecting and/or quantifying the translated SNP polypeptide.

2) Detection of Expressed Protein

The polypeptide(s) encoded by the SNP can be detected and/or quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the SNP polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to a domain of the antibody.

In preferred embodiments, the SNP polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (SNP polypeptide). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (SNP polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind SNP polypeptide(s), either alone or in combination. In the case where the antibody that binds SNP polypeptide is not labeled, a different detectable marker, for example, an enzyme-labeled antibody capable of binding to the monoclonal antibody which binds the SNP polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds SNP polypeptides is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced as described below.

3) Antibodies to SNP Polypeptides.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides encoded by the SNPs.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. Also, in this context, the term “mab's of the invention” refers to monoclonal antibodies with specificity for a polypeptide encoded by the SNP.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Pre-Screening for Agents that Bind SNP1 or SNP Polypeptide

In certain embodiments it is desired to pre-screen test agents for the ability to interact with (e.g. specifically bind to) an SNP nucleic acid or polypeptide. Specifically, binding test agents are more likely to interact with and thereby modulate expression and/or activity of the polypeptide comprising the SNP. Thus, in some preferred embodiments, the test agent(s) are pre-screened for binding to SNP nucleic acids or to SNP proteins.

In one embodiment, such pre-screening is accomplished with simple binding assays. Means of assaying for specific binding or the binding affinity of a particular ligand for a nucleic acid or for a protein are well known to those of skill in the art. In preferred binding assays, the SNP protein or nucleic acid is immobilized and exposed to a test agent (which can be labeled), or alternatively, the test agent(s) are immobilized and exposed to an SNP protein or to a SNP1 nucleic acid (which can be labeled). The immobilized moiety is then washed to remove any unbound material and the bound test agent or bound SNP nucleic acid or protein is detected (e.g. by detection of a label attached to the bound molecule). The amount of immobilized label is proportional to the degree of binding between the SNP protein or nucleic acid and the test agent.

### EXAMPLES

The following examples are offered to illustrate, but not to limit the claimed invention.

### Example 1

### A Chromatin-Integrated ErbB2 Promoter-Reporting Whole Cell Assay for High-Throughput Screening and Detection of Compounds with Potential ErbB2 Promoter Silencing Activity

Exploring various promoter silencing strategies to treat ErbB2/HER2 amplified and overexpressing human cancers, we developed a whole cell high-throughput screening assay to identify lead compounds capable of both cell permeability and ErbB2 promoter silencing. Since a transiently transfected ErbB2 promoter-reporter does not exhibit the same chromatin organization and trichostatin A (TSA) responsiveness as an endogenously integrated and amplified ErbB2 promoter, we developed stable breast cancer sublines bearing genomically integrated copies of the ErbB2 proximal promoter (0.5 kb; R06) driving expression of a short half-life luciferase reporter (R06pGL-luc) product detectable by the Promega Steady-Glo reagent (see, e.g., Example 3).

To be able to detect ErbB2 promoter silencing independent of drug-induced growth inhibition or cytotoxicity, R06pGL-luc transfected and overexpressing subclones were isolated from an ErbB2-independent breast cancer parental line, MCF-7. Characterization of one subline (MCF/R06-luc9) showing high level luciferase production within 48 h of plating in a 96-well microtitre format revealed, by DNA restriction and Southern blot analysis, faithful multicopy genomic integration of the ErbB2 promoter-reporter.

As a positive assay control, plated MCF/R06-luc9 cells were treated with TSA at a dose (<1 μM) that does not impair cell viability within 24 h measured by the Sigma MTT assay. Despite increasing R06pGL-luc ErbB2 promoter activity in transiently transfected SKBr3 cells, TSA dramatically reduces endogenous ErbB2 transcript and protein levels in these amplified and overexpressing SKBr3 cells, comparable to its inhibitory effect on luciferase expression in the MCF/R06-luc9 reporter cells. In the micrititre format MCF/R06-luc9 cells were also treated (24 h) with the NCI/DTP Diversity Set of 1,990 compounds.

At a 50 μM dose, 1.5% of compounds increased and 23% decreased reporter activity beyond the threshold level of >50% change in luciferase expression. Achieving these threshold luciferase reductions at 5 μM, 70 compounds were then tested at 500 nM and 50 nM for luciferase reducing activity and retained cell viability (MTT assay). At least 2 compounds have been identified with potential ErbB2 promoter silencing activity comparable to TSA; and by the COMPARE algorithm these two Diversity Set compounds also show a 0.34 pairwise correlation coefficient with each other for cytotoxic activity (LC50) against the NCI/DTP human tumor cell line (n=60) screen.

### Example 2

### Use of Histone Deacetylase Inhibitors to Transcriptionally Repress Endogenous Genomic and/or Chromatinized (Histone-Containing) Promoters Such as that Driving the Amplified and Overexpressed HER2/ErbB2/Neu Oncogene

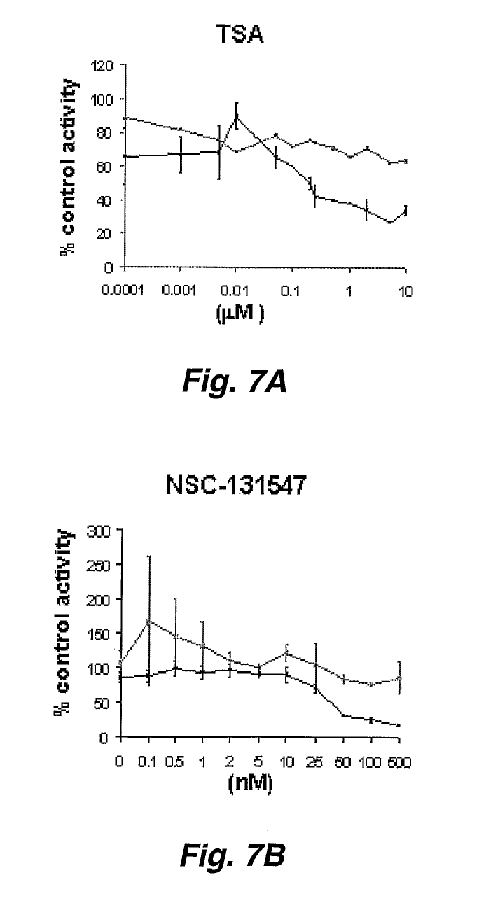

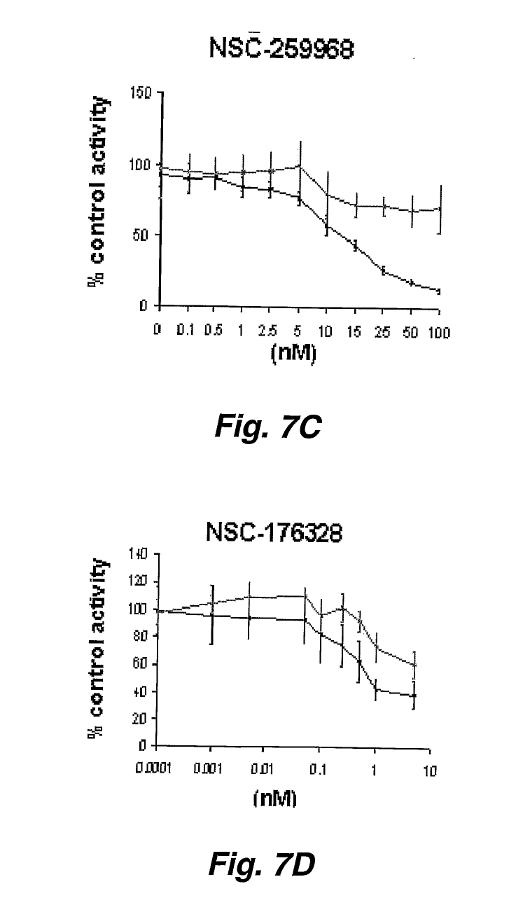

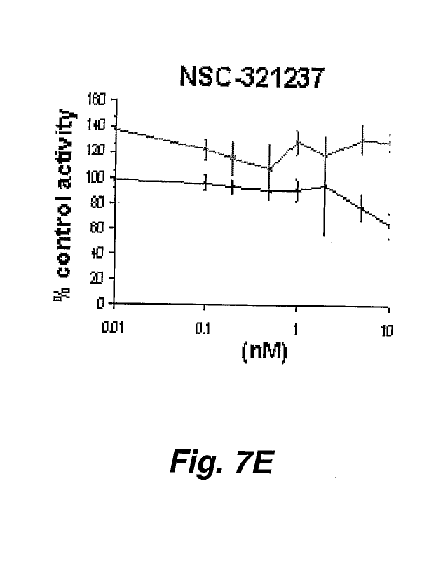

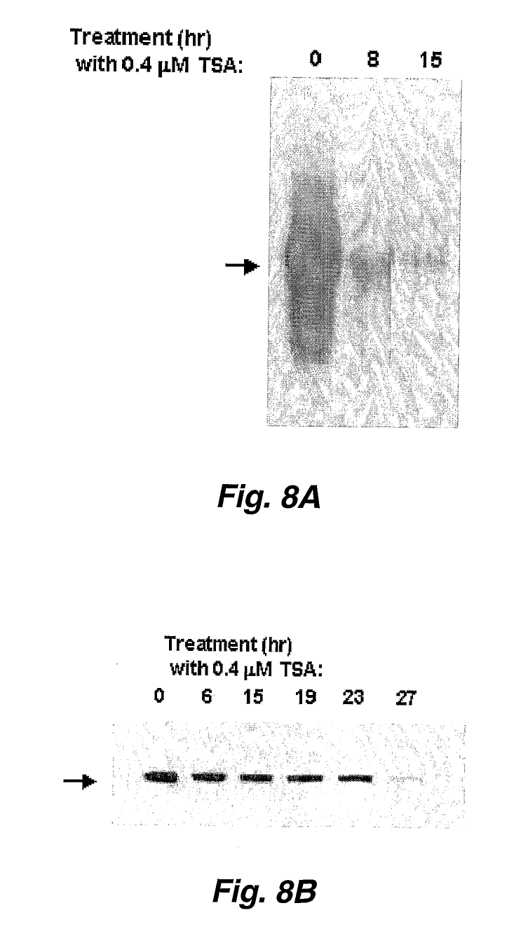

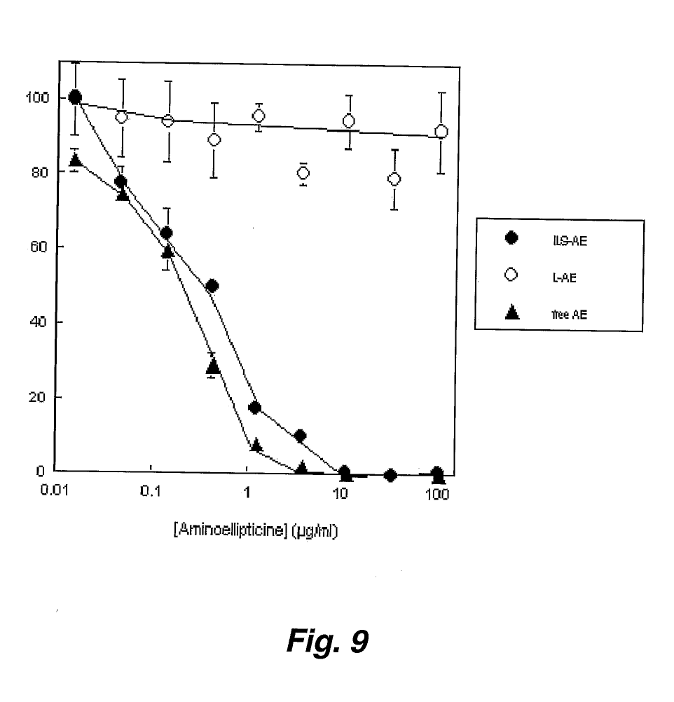

We have demonstrated that histone deacetylase (HDAC) inhibitors like sodium butyrate and trichostatin A (TSA), in a time and dose dependent fashion can silence genomically integrated and/or amplified/overexpressing promoters, such as that driving the HER2/ErbB2/neu oncogene, resulting in inhibition of gene products including transcripts and protein, and subsequent production of tumor/cell growth inhibition, apoptosis and/or differentiation (see, e.g., FIGS. 7A-7E, 8A and 8B, and 9).

HDAC inhibitors ability to silence such promoters may work either by directly altering the promoter's chromatin structure (e.g. localized histone acetylation) or by modifying acetylated non-histone proteins that bind to and regulate transcription off that promoter (e.g. Ets factors or components of the basal transcription machinery). This therapeutic promoter repressing mechanism is also paradoxical to the stimulatory response observed with these same HDAC inhibitors on gene expression constructs introduced transiently or on other endogenously integrated and chromatinized promoters such as the promoter for the acetylated Ets factor, ESX. It also appears to be more selective for certain gene transcripts since HDAC inhibitors like TSA preferentially repress the full-length (4.8 kb) vs. a truncated and alternatively processed (2.1 kb) ErbB2 transcript, which also suggests a selective inhibitory effect that is specific for certain promoter structures since the former transcripts are thought to be initiated at a more upstream ErbB2 promoter site (−69 bp) than the later transcripts (+1 bp) and are also thought to be more reflective of transcripts arising following ErbB2 gene amplification and overexpression.

Early clinical studies with HDAC inhibitors as anticancer agents appear promising (e.g. Pfizer's CI-994, MSKCC's SAHA, etc.) and are thought to relate to increased accumulation of acetylated histones; but our findings suggest that HDAC inhibitors are particularly useful in the treatment of amplified and overexpressing ErbB2 tumors where their antitumor effects may be more related to accumulation of acetylated non-histone proteins.

### Example 3

### Preparation of a Mammalian Cell Comprising a Stably Integrated Chromatinized HER2/Reporter Construct

A HER2 promoter/luciferase reporter construct was prepared using the R06 (500 bp Sma-Sma HER2 promoter fragment as described by Scott et al. (1994) J. Biol. Chem. 269: 19848-19858) coupled to the pGL3Basic luciferase reporter vector (EW1751, Promega, Inc.) according to the methods provided with the vector.

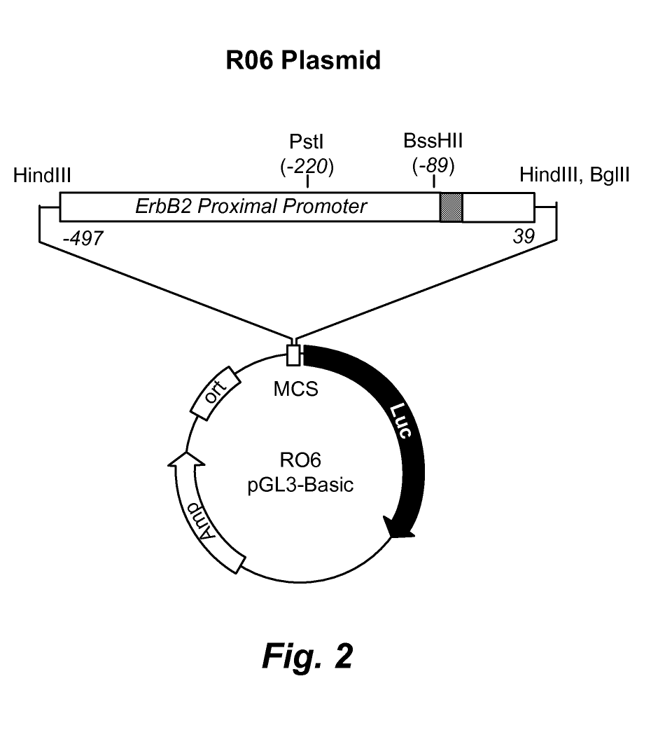

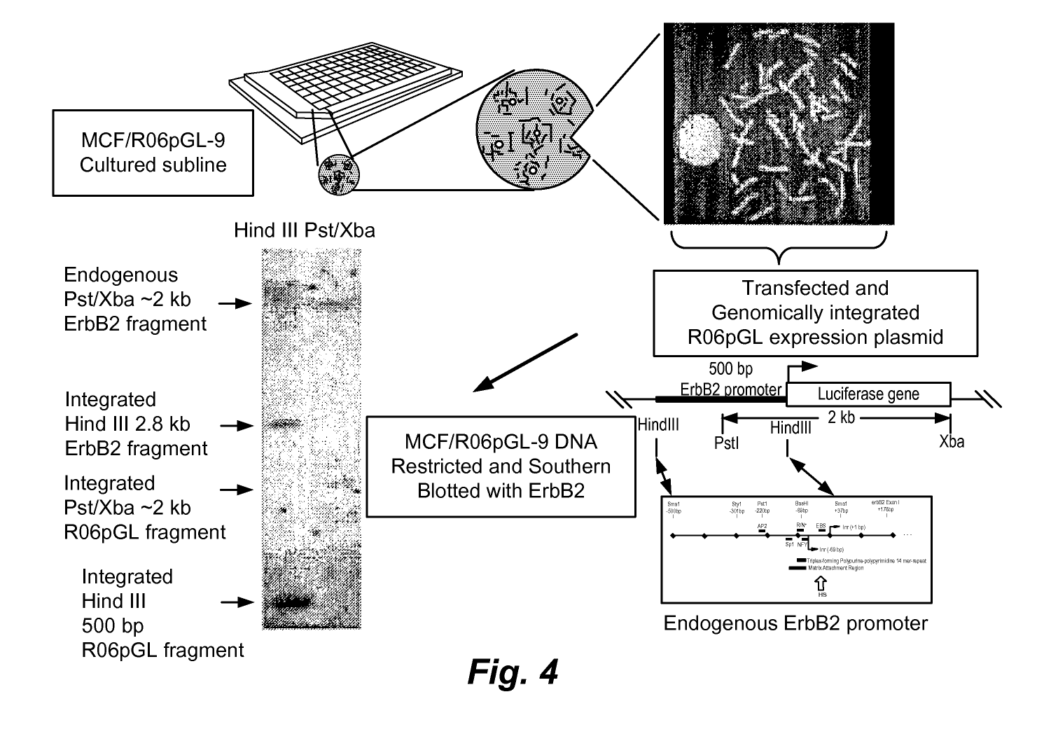

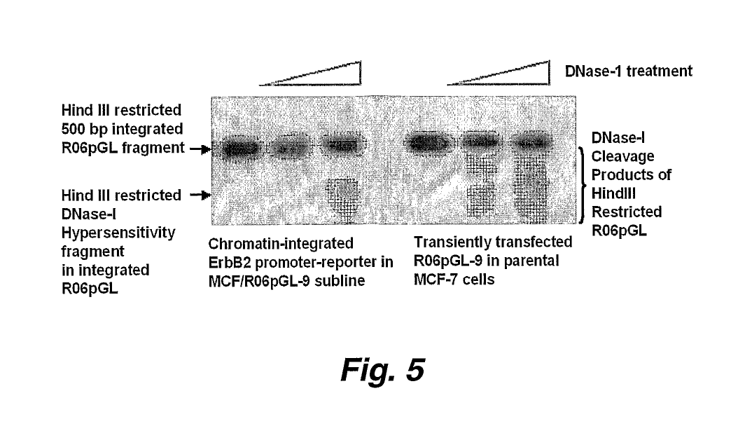

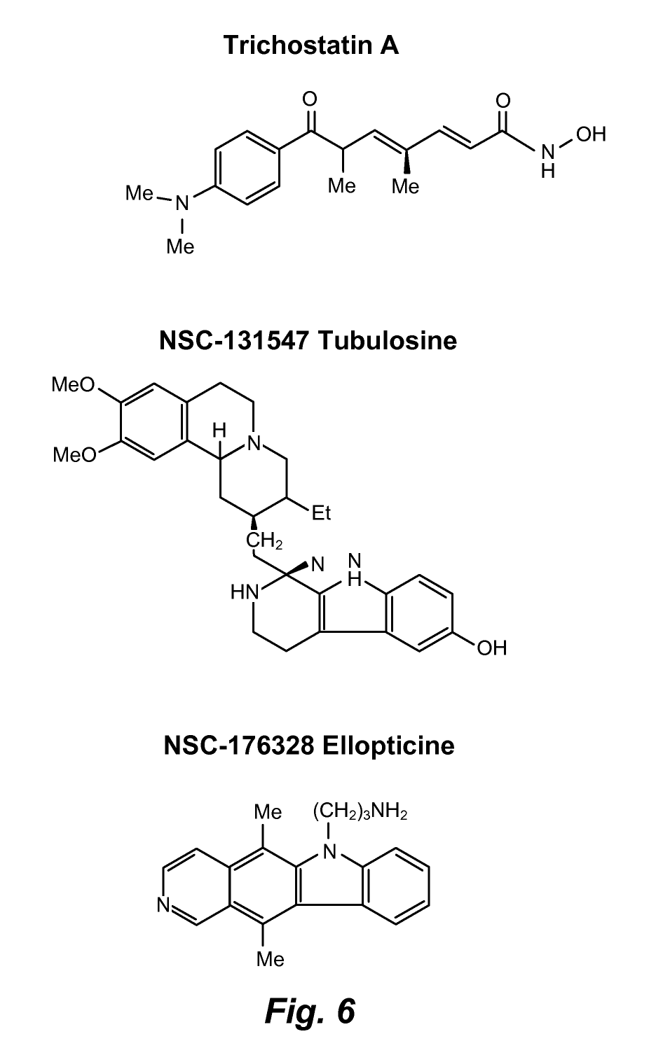

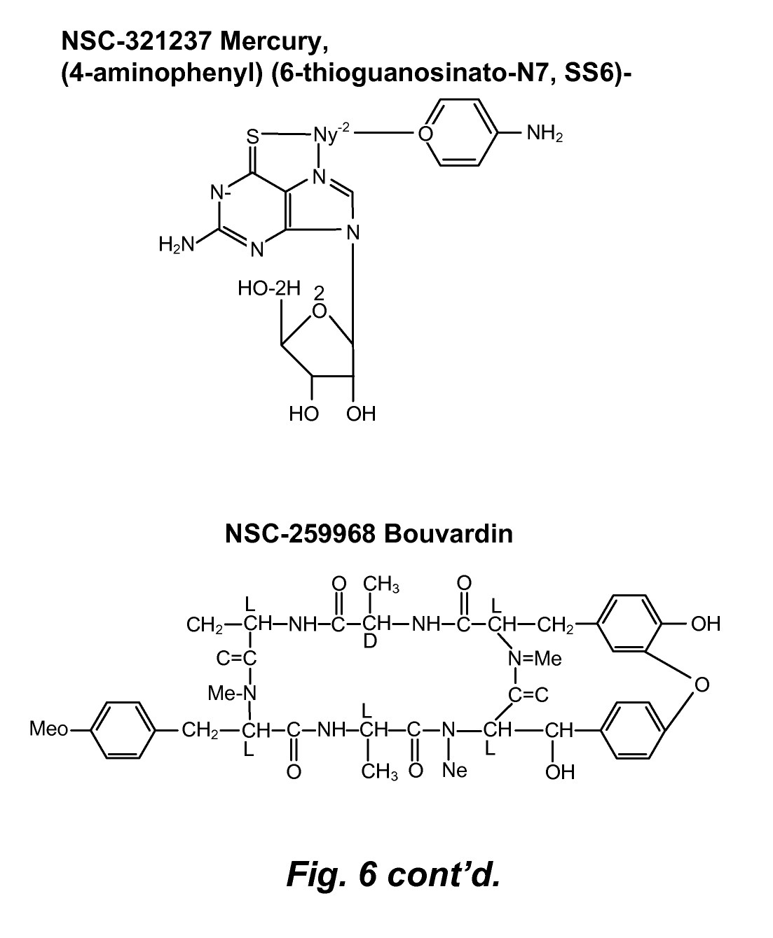

Parental cell lines MCF-7, and MDA-453 were transfected with the construct Ro6pGL (FIG. 2) in conjunction with pcMneo construct using lipid-based transfection (Effectene) at a ratio of 20:1 reporter:selectable marker (see, FIG. 4). Monoclonal and polyclonal populations were selected in 0.5 mg/ml G418 over 2 to 4 weeks.

Reporter activity was assayed as follows: From T-150 or T-75 culture flasks, about 2×10⁶ cells/well were plated into a 96 well plate format for high-throughput screening. 24 hours post plating a test agent e.g. a drug from a combinatorial library, was added to the wells at various concentrations. Certain wells were used as controls (e.g. PBS or DMSO vehicle only). Replicate wells (6 to 6 replicates per drug concentration) were run. 24 hours after addition of the test agent the cells were lysed, a luciferase reagent was added and activity in each well was read using a high-throughput screening plate reader (labsystems Fluoroskan). Parallel plates were run for cell viability using an MTT assay (Sigma).

Stable MCF7b populations were characterized as follows:

RO6 basal promoter activity in monoclonal populations ranged from <0.5 to 963 luciferase units/μg protein (presumably due to integration-site specific effects on transcription). 6/13 clones had reporter gene activity of <0.5 (possibly due to non-integration of RO6 with pcMneo). pGL3Basic basal promoter activity ranged from <0.5 to 0.53 with 8/9 clones having reporter gene activity of <0.5.

Elf-1, Notch, ESX, and ESX-Notch introduced by lipid-based transient transfection had no reproducibly significant transactivational effect on basal activity of stable RO6 populations.

No increase in promoter activity was observed on treatment of MCF-7b stable RO6 clone and poly clonal populations grown in serum-free media with 10 nM TPA or trichostatin for 24 hours (in absence of serum), rather, decreased basal promoter activity was observed in TPA and trichostatin treated samples.

Stable MDA-453 populations were characterized as follows:

RO6 basal promoter activity in monoclonal populations ranged from <0.5 to 2295 luciferase units/μg protein (presumably due to integration-site specific effects on transcription). 14/32 clones had reporter gene activity of <0.5; GL3Basic basal promoter activity ranged from <0.5 to 0.293, with 8/9 monoclonal populations being <0.5.

Elf-1 introduced by lipid based transient transfection had no reproducibly significant transactivational effect on MDA-453 stable RO6 populations. The effect of Notch was also investigated, also with no significant transactivational effect. ESX introduced by retroviral infection (supernatant infection using supernatant generated from Phoenix producer cells transiently transfected with an MXI EGFPhESX construct had no reproducibly significant transactivational effect on MDA-453 stable RO6 polyclonal populations.

TPA treatment (10 nM TPA in DMSO, 8 and 48 hr timepoints) of two MDA-453 RO6 polyclonal populations (in the presence of serum) had no significant effect on basal promoter activity (compared with DMSO only).

In the micrititre format MCF/R06-luc9 cells were also treated (24 h) with the NCI/DTP Diversity Set of 1,990 compounds (see Example 2). At least 2 compounds have been identified with potential ErbB2 promoter silencing activity comparable to TSA; and by the COMPARE algorithm these two Diversity Set compounds also show a 0.34 pairwise correlation coefficient with each other for cytotoxic activity (LC50) against the NCI/DTP human tumor cell line (n=60) screen.

### Example 4

### Transcriptional Repression of ErbB2 by Histone Deacetylase Inhibitors Detected by A Genomically Integrated ErbB2 Promoter-Reporting Cell Screen

The antitumor activity of histone deacetylase (BOAC) inhibitors has been linked to gene expression induced by acetylation of histone and non-histone proteins; but the molecular basis for their antitumor selectivity remains largely unknown. With development of a genomically integrated ErbB2 promoter-reporting breast cancer cell screen, ErbB2 promoter inhibiting activity was observed by the HDAC inhibitors trichostatin A (TSA) and sodium butyrate. Paradoxically, these agents stimulated the episomal form of this ErbB2 promoter-reporter introduced by transient transfection. Transcriptional run-off assays in ErbB2 amplified and overexpressing breast cancer cells confirmed that within 5 hours, TSA exposure profoundly inhibits ErbB2 transcript synthesis off the amplified oncogene yet preserves transcription off single copy genes like the epithelial-specific Ets family member, ESX. Northern analyses of ErbB2 overexpressing breast cancer lines (SKBR3, BT-474, MDA-453) showed that within 24 hours of submicromolar treatment by TSA, ESX transcript levels increase while ErbB2 transcript levels rapidly decline, with no TSA affect apparent on the open chromatin configuration of either gene as monitored by DNase I hypersensitivity. Actinomycin D studies confirmed that in addition to inhibiting ErbB2 transcript synthesis, TSA selectively destabilizes mature ErbB2 transcripts enhancing their decay. While TSA markedly reduced ErbB2 protein levels in these overexpressing cell lines, TSA treatment of MCF/HER2-18 cells engineered to overexpress the ErbB2 receptor under control of a heterologous promoter increased their expression of ErbB2 protein. These findings suggest that further studies are warranted to determine if ErbB2-positive human cancers represent unusually sensitive clinical targets for HDAC inhibitor therapy.

### Introduction.

Despite recent approval of an anti-ErbB2 therapeutic antibody (trastuzumab) to treat advanced breast cancer and the clinical promise of even newer ErbB2 receptor-targeted therapeutics (1,2), there is increasing interest in erbB2 oncogene-silencing strategies because the amplified oncogene and its expressed transcripts per tumor cell are far fewer in copy number than the overexpressed ErbB2 protein product. In addition, the prevalence of obvious resistance mechanisms to ErbB2 receptor-based therapy points to the clinical need for alternative anti-ErbB2 strategies and combinatorial approaches (1).

Antisense and ribozyme strategies have proven partially successful at downregulating ErbB2 transcript and protein expression in preclinical models, but have so far failed to enter clinical trials (3-6). Efforts to target the 2-to-10-fold amplified copies per tumor cell of the erbB2 oncogene have also been explored. EIA induced repression of the ErbB2 promoter has already entered clinical trials but its CBP/p300 mediated repression mechanism is not specific to the ErbB2 promoter and this therapeutic requires efficient intratumor gene delivery and expression (7). Other more specific ErbB2 promoter-targeting approaches which have shown promise in vitro but have not yet been evaluated in vivo include ErbB2 promoter-binding and triplex-forming oligos (8), polyamides with nanomolar affinity for the ErbB2 promoter's Ets binding site (EBS) (9), and EBS-targeted chimeric transcriptional repressors (10). It is expected that virtually all of these ErbB2 transcript- and promoter-targeted strategies are compromised most by their limited in vivo bioavailability and/or solid tumor uptake, and also face significant intracellular and intranuclear delivery and trafficking challenges prior to their clinical advancement (10).

Exploring additional ErbB2 promoter-silencing strategies not encumbered by the above intratumor delivery and intranuclear distribution limitations, we developed a whole-cell high-throughput screen to identify cell permeable small molecule inhibitors of the ErbB2 promoter. To this end, stable transfection of the ErbB2-independent breast cancer cell line MCF-7 was undertaken to produce a subline (MCF/R06pGL-9) bearing a genomically integrated and chromatinized ErbB2 proximal promoter construct driving a luciferase reporter gene for use in high-throughput screening of chemical libraries for compounds capable of inhibiting ErbB2 driven luciferase activity without producing general cytotoxicity (as measured by MTT viability assay). Using this screening assay the histone deacetylase (HDAC) inhibitors sodium butyrate and trichostatin A (TSA) were identified as potent and relatively specific ErbB2 promoter inhibiting agents. After validating their ErbB2 promoter and transcript inhibiting potential against a panel of breast cancer cell lines that endogenously overexpress ErbB2, we also observed that HDAC inhibitor treatment selectively destabilizes preexistent ErbB2 transcripts leading to an accelerated loss of intracellular ErbB2 mRNA and protein.

### Methods.

Drugs, Breast Cancer Cell Lines, Probes and Antibodies.

The histone deacetylase inhibitors, sodium butyrate and trichostatin A (TSA), as well as the RNA polymerase inhibitor, actinomycin D (Act D), and the ribosome translocation inhibitor, cycloheximide, were all commercially obtained (Sigma). ErbB2-independent (MCF-7) and ErbB2-dependent/overexpressing (SKBR3, BT-474, and MDA-MB-453) breast cancer cell lines were originally obtained from American Type Culture Collection and were passaged in tissue culture as recently described (10). MCF7/HER2-18 were derived by stable transfection into MCF-7 cells of a human HER2/ErbB2 cDNA (coding region only) within a 4.7 kb pRK5 expression plasmid downstream and under the control of a CMV promoter/enhancer and with SV40 termination and polyadenylation signals, as has been previously described (11). ErbB2 genomic and cDNA probes used for Southern and Northern blots and transcriptional run-off slot-blots have been described (11-13); monoclonal antibody to ErbB2 protein used for Western blots was commercially obtained (Calbiochem). ESX genomic and cDNA probes used for Southern and Northern blots and transcriptional run-off slot-blots, as well as the anti-ESX polyclonal antibody used for Western blots, have all been described (14,15). Monoclonal antibody to a tubulin used on Western blots was commercially obtained (Calbiochem).

MCF/R06pGL-9 Construction and Drug Screening

A luciferase reporter plasmid (designated RO6pGL-luc) was constructed by inserting the ˜500 bp SmaI-SmaI fragment of the human ErbB2 proximal promoter (12) into the pGL3Basic plasmid driving a luciferase reporter gene, as has been described (10). R06pGL-luc and a plasmid expressing neomycin phosphotransferase were co-transfected into the MCF-7 cells (ATCC) using Effectene (Qiagen). Cells were selected for stable expression of luciferase and neomycin (G418)-resistance. Single cell clones including MCF/R06pGL-9 were isolated and maintained in DME H-16 medium supplemented with 10% fetal bovine serum, 10 μg/ml insulin, 100 μg/ml penicillin/streptomycin, and 500 μg/ml G418. MTT cell viability and luciferase expression assays on the MCF/RO6pGL-9 sublime were performed after plating the subline into 96-well culture dishes at a density of 10⁴ cells/well in 100 μL medium. After 24 hour cell plating, TSA from stock DMSO solution was diluted into 100 μL medium and then added into eight replicate wells at the indicated concentrations. After 24 hours of drug (or 0.5% DMSO vehicle control) treatment, 3-[4,5-dimethylthiazol-2-yl]-2,5-diphenyltetrazolium bromide (MTT, Sigma) was added to the cells to a final concentration of 0.5 mg/mL and incubated at 37° C. for 4 hours. The medium was carefully aspirated. The colored formazan product was solubilized in 100 μL of 0.1 N HCl in isopropanol. The reaction was quantified by absorbance at 570 nm measured with a microplate reader (Molecular Devices) and the mean (±SD) optical density recorded after normalization by the vehicle treated controls. In parallel, plated and drug treated cells were washed once with phosphate-buffered saline and lysed for 15 minutes at room temperature in lysis buffer (Promega), and luciferase activity measured immediately from the cell extract by commercial assay kit (Promega) and using a microplate luminometer (LabSystems), with results similarly expressed as the mean ((SD) of control activity after normalization for vehicle treated controls. The <3 hour intracellular half-life of the luciferase product from the R06pGL reporter construct detected by this commercial luminescence assay allows for rapid and sensitive detection of virtually complete (>8 half-life reduction in reporter activity) inhibition of the intracellular ErbB2 promoter within 24 hours of drug treatment.

DNase I Hypersensitivity and Southern Blotting, Northerns and Transcriptional Run-Off Slot-Blotting, and Western Immunoblotting

The conserved and singular DNase I hypersensitivity sites found in the proximal ErbB2 and ESX promoters (12,15) were assayed as before (12). Briefly, following culture treatment of cells, nuclei isolated by mild detergent lysis in a buffer containing 10 mM Hepes pH 7.9, 1.5 mM MgCl₂, 10 mM KCl, 0.4% NP-40, 10% glycerol and 1 mM DTT. Partial DNase I digestions were carried out by varying DNase I treatment times (0-15 minutes at 37( ) at a fixed DNase I concentrations (0.5-1.0 units per 10⁶ nuclei). After purifying and restricting the DNA, it was electrophoresed through 0.7% agarose gels and blotted onto nylon membranes, UV-crosslinked and hybridized with randomly ³²P-labelled ErbB2 and ESX promoter probes, as similarly performed for routine Southern blotting. For Northern blotting, total (treated vs. control) cell RNA (10 μg/sample lane) was isolated using TRIzol (Invitrogen) according to manufacturer's specifications, electrophoresed into 1% agarose-formaldehyde gels and transferred onto membranes that were then hybridized with ³²P-labelled ErbB2 and ESX cDNA probes. For transcriptional run-off assays cell nuclei were first isolated from treated and control cells as described for the DNase I studies. Elongatation of initiated nascent RNA chains was performed at 37 (C for 30 minutes using ˜5×10⁶ nuclei per reaction in a buffer containing 10 mM Tris pH7.5, 2.5 mM MgCl₂, 150 mM KCl, 1 mM DTT, 10% glycerol, 0.5 mM ATP, GTP and CTP, and 100 μCi of [α-³²P]UTP (800 Ci/mmol). Nuclear RNA was purified by the addition of 100 units of RNase-free DNase I (Roche) per reaction for 2 minutes followed by TRIzol processing. Radiolabelled RNA was hybridized (˜4×10⁶ cpm) at 68 (C in ExpressHyb (Clontech) for 24 hours to nylon filter membranes previously slotted with 0.5 μg per slot of unlabelled ErbB2 cDNA fragments (from either the transmembrane or C-terminal domains), ESX cDNA fragments, and empty plasmid control, as we have previously described (16). Filters were washed at 64 (C in 0.2 SSC and 0.5% SDS. For Western blotting, whole-cell extracts from control vs. treated cells were boiled in sample loading buffer (1% SDS, 20% glycerol, 100 mM DTT, 50 mM Tris, pH 6.8), gel lanes loaded for constant total protein (15 μg), electrophoresed into 9% sodium dodecyl sulfacte polyacrylamide (SDS-PAGE) gels, transferred onto membranes (Immobilon-P, Millipore), and the protein-bound membranes hybridized with a primary antibody followed by a horseradish peroxidase-conjugated secondary antibody (Sigma), and specific protein bands visualized by chemiluminescent substrate (Pierce), as previously described (10).

### Results.

Genomically Integrated vs Episomal ErbB2 Promoter Constructs

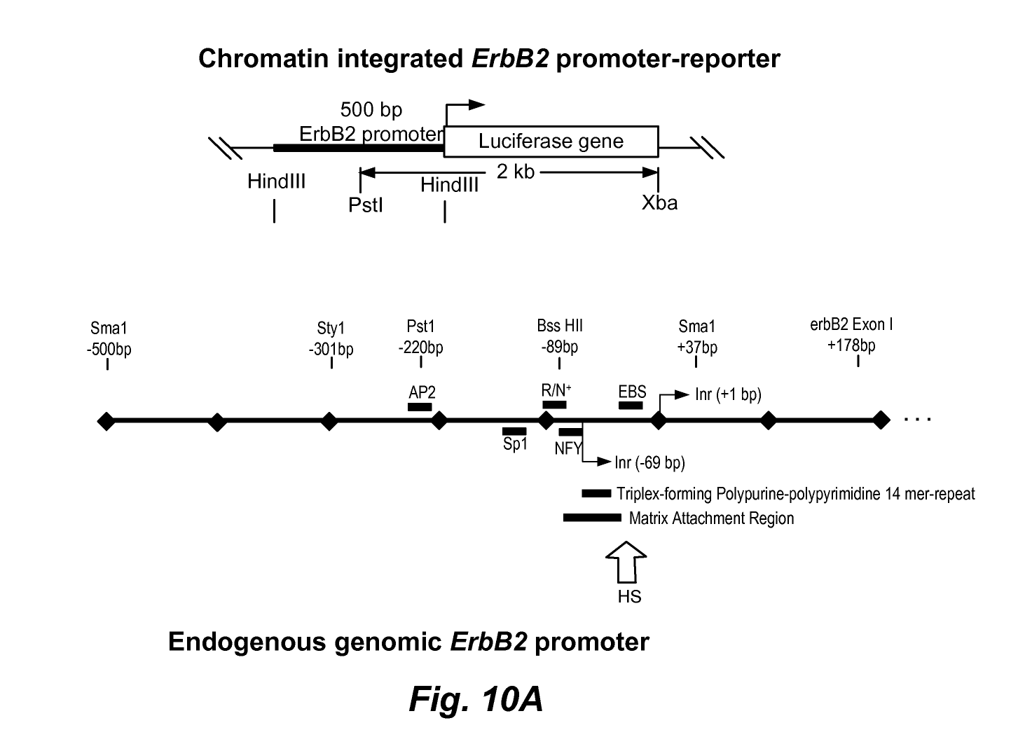

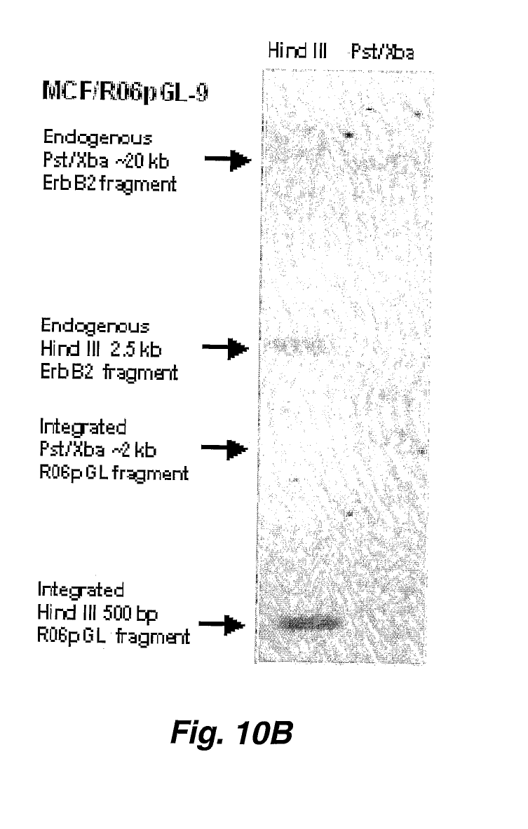

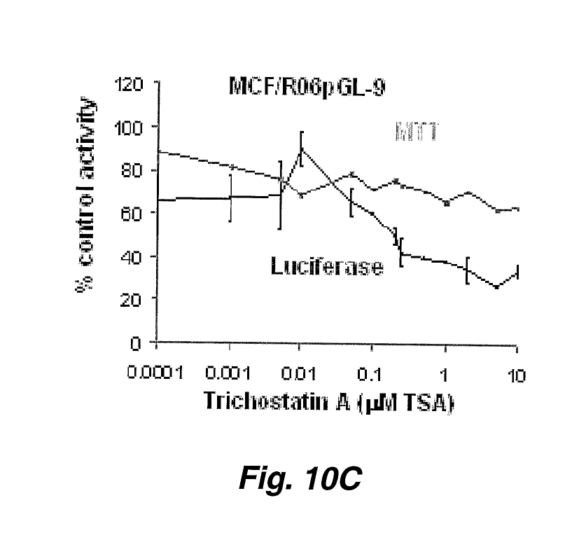

To establish an assay system capable of reflecting endogenous ErbB2 promoter activity in human breast cancer cells lines, stable integration of an ErbB2 promoter-luciferase reporter construct (R06pGL) into various breast cancer cell lines was undertaken. ErbB2 amplified and overexpressing SKBR3 and MDA-453 cells and the low ErbB2 expressing MCF-7 cells were co-transfected with both a 500 bp ErbB2 promoter-driven luciferase construct (FIG. 10) and a Neo (G418) selection plasmid. Stable integration of the ErbB2 promoter-reporter was successfully achieved only in the MCF-7 cell line, producing the clonally isolated MCF/R06pGL-9 subline with faithful integration of the 500 bp ErbB2 promoter-reporter documented by Southern blot analysis. (FIG. 10). No luciferase expressing clones could be isolated from multiple transfection attempts into SKBR3 cells; and multiple luciferase expressing MDA-453 clones showing very slow initial growth all reverted to wildtype culture growth in association with loss of their genomically integrated 500 bp ErbB2 promoter-driven reporter constructs (data not shown).

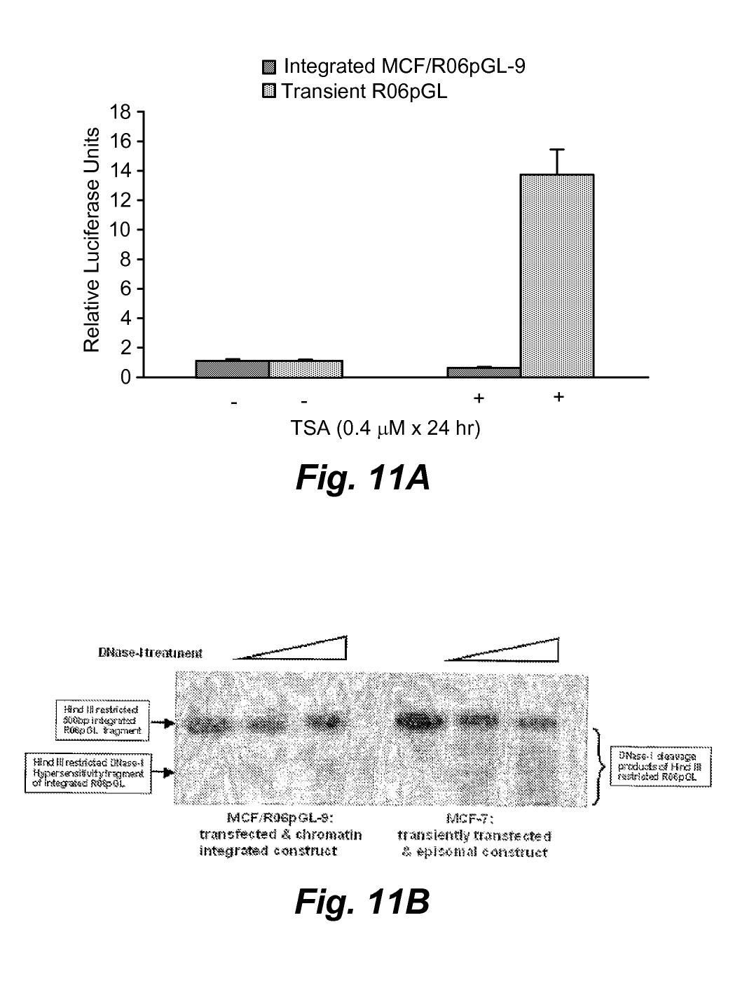

Using the genomically integrated ErbB2 promoter-reporter subline MCF/RO6pGL-9 in a 96 well high-throughput screening (HTS) format to begin screening chemical libraries (e.g. the NCI/DTP Diversity Set), we were surprised to observe that the histone deacetylase (HDAC) inhibitor trichostatin A (TSA) resulted in significant reduction of luciferase activity with little impact on cell viability (assess by MTT assay) following 24 hours of culture exposure to TSA concentrations up to 50 μM (FIG. 10). In an episomal context and following transient transfection of the RO6pGL construct into MCF-7 cells, TSA produced diametrically opposite (>10-fold stimulatory) effects on this same ErbB2 promoter-driven reporter (FIG. 11). Southern blot comparisons of DNase I treated MCF/R06pGL-9 cell nuclei and those from transfected MCF-7 cells bearing the episomally introduced R06pGL plasmid confirmed the lack of a discrete DNase-I hypersensitivity site in the episomal R06pGL and the presence of such a site in the genomically integrated R06pGL, similar in location and intensity to that found within the endogenous ErbB2 promoter (FIG. 11). The strong TSA stimulatory effect on the transiently transfected ErbB2 promoter-reporter appeared independent of the amount of R06pGL plasmid introduced (1 ng-1 μg), and was similarly observed with transient transfection and TSA treatment of ErbB2 overexpressing SKBR3 and MDA-453 cells (data not shown).

Downregulation of Endogenous ErbB2 Transcripts by TSA

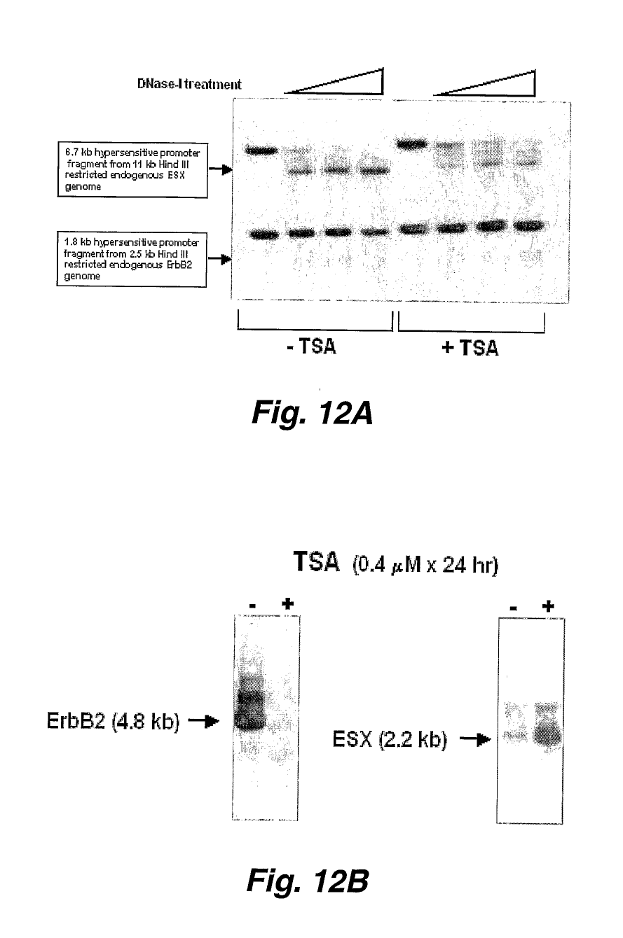

Northern analyses were performed to determine the influence of TSA upon endogenous ErbB2 transcript levels. Gels were normalized for constant rRNA loading and the effect of TSA treatment on long-lived 4.8 kb ErbB2 transcript levels were compared with its effects on the short-lived 2.2 kb transcripts of ESX, an epithelial-specific Ets transcription factor often co-expressed with ErbB2 in human breast cancer cell lines (14). FIG. 12 demonstrates the near total disappearance of ErbB2 transcripts in SKBR3 cells after 24 hours of TSA treatment, associated with a simultaneous 5-fold increase in ESX transcript levels in these treated cells. Given these opposite TSA effects on ErbB2 and ESX transcript levels, and a previous report suggesting that TSA activates transcription in association with its enhancement of DNase I hypersensitivity in a gene's regulatory locus (18), we were also surprised to observe the lack of any TSA treatment effects on the hypersensitive loci within either the ErbB2 or ESX promoters (FIG. 12). Virtually complete elimination of ErbB2 transcript levels was also observed following similar TSA treatment of MDA-453 and BT-474 cells; and could also be induced by treatment with sodium butyrate (3 mM×24 hours), another well known HDAC inhibitor (data not shown).

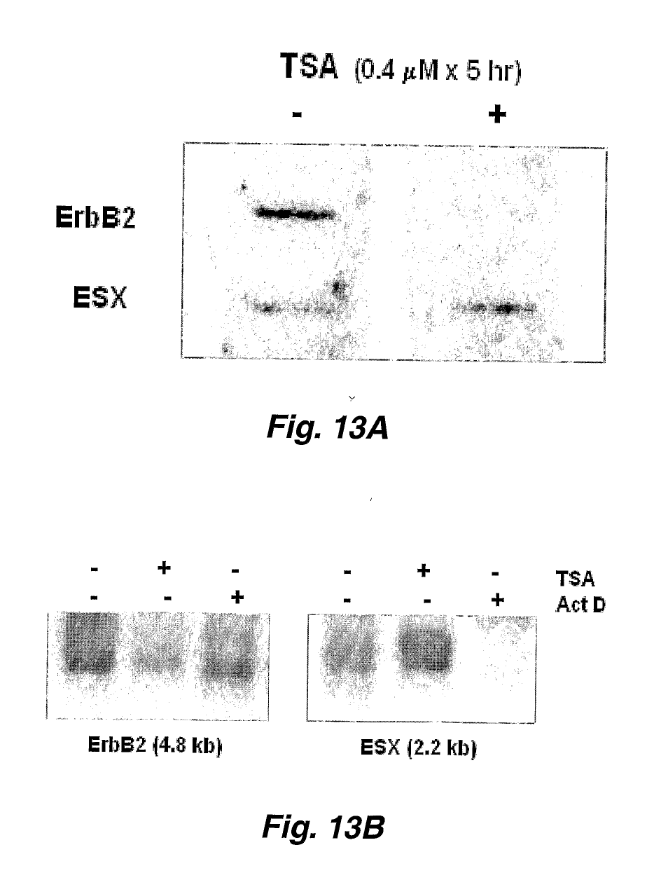

Nuclear run-off experiments were performed on ErbB2 overexpressing breast cancer cells to confirm the conclusion drawn from TSA induced downregulation of luciferase expression in MCF/R06pGL-9 cells that HDAC inhibitors can suppress ErbB2 promoter activity and presumably also repress ErbB2 transcription. Nuclei from SKBR3 cells treated for 5 hours with TSA were isolated and their nascent nuclear transcripts elongated in the presence of radiolabelled nucleotides; the labelled RNA was isolated and hybridized to membranes slotted with ESX cDNA and ErbB2 cDNA fragments, including those from either the carboxy-terminus or transmembrane domain to assure good transcript representation from this ˜30 kb oncogene. Slot blot hybridization stringency was adjusted so that no signal could be detected from those control slots containing the empty plasmid. As shown in FIG. 13, TSA culture treatment for only 5 hours profoundly suppresses synthesis of new ErbB2 transcripts off the amplified oncogene yet preserves ESX mRNA synthesis off this single copy gene.

The rate of ErbB2 transcript loss observed in TSA treated ErbB2-positive breast cancer cells (e.g. FIG. 12) suggested a TSA induced accelerated decay of the normally long-lived intracellular ErbB2 mRNA, since an 8 hour (˜half-life of ErbB2 mRNA) TSA treatment also resulted in <20% of control ErbB2 transcript levels as detected by Northern assays (data not shown). To test this possibility, Northerns were performed on RNA isolated from SKBR3 cells after 5 hours treatment with either actinomycin D (Act D) or TSA (0.05 or 0.40 μM). As shown in FIG. 13, TSA greatly enhanced the rate of ErbB2 mRNA decay relative to Act D treated cells (which show only a slight reduction in ErbB2 mRNA level), yet this exposure to Act D was sufficient to fully inhibit transcription and result in the complete loss of the short-lived ESX mRNA. Of interest, a 5 hour SKBR3 treatment with either 0.05 μM or 0.40 μM TSA dose produced comparable reductions in Northern blot ErbB2 transcript levels (data not shown); this apparent plateau in the 5 hour TSA dose response suggests that any observed decline in total ErbB2 transcript levels likely reflects at least two independent CxT responses to TSA, one for its inhibition of transcript synthesis and another for its acceleration of ErbB2 transcript decay. TSA treated BT-474 and MDA-453 cells showed a similarly accelerated loss of ErbB2 transcript levels as compared to their treatment with Act D (data not shown). In additional experiments to explore the nature of this TSA induce post-transcriptional decay of ErbB2 transcripts, SKBR3 cells were treated with a dose of cycloheximide sufficient to block mRNA ribosomal translocation (50 μg/ml), and while this was observed to have no effect on ErbB2 transcript levels in control cells it completely inhibited the accelerated ErbB2 mRNA degradation induced by TSA. However, a similar cyclohexamide treatment had no ability to arrest TSA induced ErbB2 transcript decay in BT-474 or MDA-453 cells (data not shown). These observations indicate that the unknown mechanism(s) underlying TSA induced ErbB2 mRNA decay may be distinct in different cell lines and likely independent from those underlying the inhibition of ErbB2 transcript synthesis.

Downregulation of Endogenous ErbB2 Protein by TSA

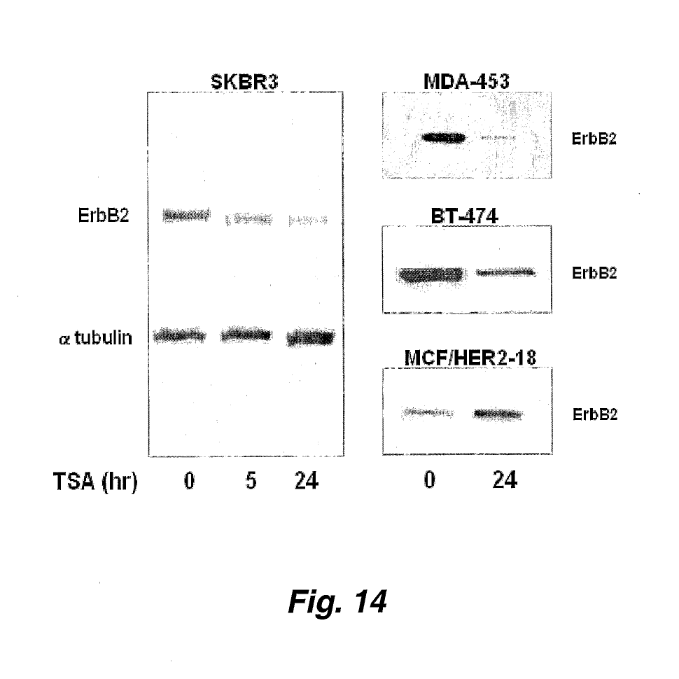

That a genomically integrated but not episomal ErbB2 promoter-reporter construct correctly recapitulated the endogenous ErbB2 promoter's response to TSA underscores the need to develop ErbB2 models that better resemble the endogenously overexpressed oncogene. In this regard, TSA also failed to repress (and actually stimulated) ErbB2 overexpression in another MCF-7 subline, MCF/HER2-18, engineered to transcriptionally overexpress ErbB2 and its functional 185 kDa surface receptor, and now commonly used to evaluate novel ErbB2 receptor-targeted therapeutics (11,20,21). The ectopically introduced and genomically integrated ErbB2 expression construct in this subline was placed under the control of a CMV promoter and contains only the ErbB2 protein coding sequences fused to an SV-40 polyadenylation signal (11). As shown by the Western analyses in FIG. 14, 24 hour TSA treatment of SKBR3, MDA-453 and BT-474 cells produces the expected marked decline in their endogenous 185 kDa ErbB2 protein levels (normalized to α-tubulin levels). In contrast, the level of ectopic 185 kDa ErbB2 protein overexpressed in MCF/HER2-18 cells is actually further stimulated under these same TSA treatment conditions.

### DISCUSSION

We developed a whole-cell high-throughput screen (HTS) for cell permeable agents capable of selectively inhibiting the ErbB2 promoter without producing generalized cytotoxicity. After verifying by Southern blot and DNase I hypersensitivity assays the integrity of the chromatinized ErbB2 promoter-reporter construct integrated within the genome of the MCF/R06pGL-9 subline, the HDAC inhibiting agents TSA and sodium butyrate tested in this cell screen were identified as potent ErbB2 promoter-repressing candidates. Consistent with earlier reports questioning conclusions drawn from cell studies using non-integrated (e.g. episomal or transiently transfected) promoter-reporter constructs in favor of genomically integrated and chromatinized promoter-reporters (17), we observed significant suppression by TSA of the chromatinized ErbB2 promoter-reporter in the MCF/R06pGL-9 subline in contrast to a paradoxical >10-fold stimulating effect of TSA on parental MCF-7 cells transiently transfected with the same ErbB2 promoter-reporter construct (R06pGl). This HTS format employing a genomically integrated promoter driving a short half-life (<3 hour) luciferase reporter appears particularly useful for rapid and sensitive monitoring of drug-induced effects targeted to the regulatory region of genes like erbB2 whose endogenous transcript and protein products can be so long-lived as to require days of continuous promoter inhibition to measure significant declines in intracellular levels of these gene products.

Our HTS identification of an HDAC inhibiting effect on the chromatinized ErbB2 promoter was validated by showing that comparable submicromolar exposure to TSA significantly reduces intracellular ErbB2 mRNA and protein levels in a panel of culture treated breast cancer cell lines (SKBR3, MDA-453, BT-474) known to contain the endogenously amplified and overexpressing erbB2 oncogene. This TSA induced repression of endogenously ErbB2 expression was not due to a generalized repression of intracellular gene expression since reprobing the Northern and Western blots containing the same RNA and protein from these TSA treated cell lines revealed a strong TSA stimulating effect on the expression of other endogenous genes like the Ets family transcription factor, ESX. Given the recent report describing TSA activation of estrogen receptor (ER, α isoform) transcription in similarly treated breast cancer cells associated with TSA enhanced ER gene locus sensitivity to DNase I (18), we evaluated TSA treatment effects on the singular DNase I hypersensitivity sites associated with endogenous erbB2 and ESX promoter loci. Unlike the reported induction of ER genomic sensitivity to DNase I potentially explaining TSA stimulated ER gene expression (18), TSA produced no significant change in the prominent DNase I hypersensitivity sites of either erbB2 or ESX, suggesting that alteration of the already open chromatin configuration of these two actively transcribing genes cannot adequately explain the opposing effects of TSA on their intracellular transcript levels.

Nuclear run-off assays performed on ErbB2 overexpressing and ESX expressing SKBR3 cells confirmed that the ErbB2 transcriptional silencing effect of this HDAC inhibitor is profoundly evident within 5 hours of TSA treatment and concurrent with preserved ESX transcription. Given the well established long half-life of intracellular ErbB2 transcripts in contrast to the short (<2 hours) half-life of ESX transcripts, the observed decline in total SKBR3 ErbB2 transcript levels within 5 hours of TSA treatment suggested that HDAC inhibition might also affect the stability of mature ErbB2 transcripts in addition to inhibiting synthesis of new ErbB2 mRNA. Treating SKBR3 cells for 5 hours with an RNA polymerase inhibiting dose of Act D sufficient to completely deplete endogenous ESX transcript levels produced little detectable change in total ErbB2 transcript levels, in keeping with the long half-life of these transcripts and in contrast to the marked decline in ErbB2 transcript levels caused by TSA treatment of similar duration. Comparable results were observed in TSA and sodium butyrate treated BT-474 and MDA-453 cells, indicating that HDAC inhibitors not only repress the synthesis of endogenous ErbB2 transcripts but also accelerate the decay of preexistent mature ErbB2 transcripts. Further studies are needed to explore the likelihood that HDAC inhibitors produce reductions in total ErbB2 transcript levels based on two probably independent subcellular mechanisms, each with different dose responses to this class of drugs: one resulting in inhibition of ErbB2 transcript synthesis and another resulting in enhanced ErbB2 transcript decay.

Transcript stability is thought to be regulated by trans-acting factors that bind to cis-acting elements within untranslated regions (UTRs) in the 5′ and/or 3′ termini of mature mRNA molecules, mediating mRNA decay through poorly defined mechanisms (19). To test the dependence of ErbB2 transcriptional repression by HDAC inhibitors on regulatory elements contained within both the native ErbB2 promoter and UTRs of its endogenous transcript, we turned to another MCF-7 subline, MCF/HER2-18, engineered to overexpress ErbB2 (45-fold over parental line) and commonly used to assess the activity of ErbB2 receptor-targeted therapeutics (11,20,21). Overexpression of ErbB2 protein in MCF/HER2-18 cells results from a stably transfected and genomically integrated ErbB2 expression vector lacking the native ErbB2 promoter (replaced by a CMV promoter) and all non-coding ErbB2 cDNA sequence (ie., 5′ and 3′ UTRs). Interestingly, TSA treatment of MCF/HER2-18 cells does not repress transcription off this engineered and genomically integrated ErbB2 construct, but rather stimulates additional ectopic gene expression. This observation not only points to the limited use of such artificial cell lines to evaluate novel anti-ErbB2 therapeutics, but also suggests that the full ErbB2 promoter-repressing and transcript destabilizing activity of TSA requires both native ErbB2 promoter sequence as well as some (as yet undefined) 5′ or 3′ UTR regulatory element within the mature ErbB2 transcript.

HDAC inhibitors like sodium butyrate and the hydroxamic acid derivatives TSA and suberoylanilide hydroxamic acid (SAHA), as well as other structurally unrelated HDAC inhibitors, are known to produce in vitro and in vivo antiproliferative, differentiation- and apoptosis-inducing effects against breast and other epithelial cancers; and some of these are showing promise in early phase clinical trials (22-28). The antitumor effects of HDAC inhibitors are thought to arise from acetylation of both histones (H3, H4) and non-histone transcription-related factors (29,30), leading to enhanced expression of such cell cycle regulating genes as p21Waf1 (22,23). However, the molecular basis for the in vitro or in vivo tumor selectivity of HDAC inhibitors remains largely unknown. Microarray studies indicate that of the <10% of actively transcribing genes whose cellular expression are significantly affected by HDAC inhibitors like sodium butyrate and TSA, the vast majority are upregulated with very few genes of known function identified as being downregulated within 48 hours of treatment (31,32). Moreover, preliminary assessment of the NCI/DTP Diversity Set of nearly 2000 chemical compounds against our genomically integrated ErbB2 promoter-reporting MCF/R06pGL-9 cell screen indicates that <0.3% of potential anticancer compounds have ErbB2 promoter-inhibiting specificity and potency approaching that of HDAC inhibitors (33).

The critical growth and developmental role of normal ErbB2 as well as its amplification and overexpression during human epithelial tumorigenesis highlight the potential mechanistic and clinical significance of our observation showing selective downregulation of ErbB2 within hours after cellular exposure to an HDAC inhibitor like TSA. Recent studies reporting on HDAC inhibitor activity against human breast cancer cells did not evaluate a sufficient number of ErbB2-positive vs. ErbB2-negative cell lines to determine if ErbB2 overexpression represents a predictive tumor marker for HDAC inhibitor antitumor activity (27,28). Furthermore, the NCI/DTP's screen of TSA (NSC-709238) determined a median cytotoxic concentration (LC50) for TSA of >10 μM against their 60 human cancer cell line panel (data courtesy of J. Johnson, NCI/DTP), much higher than the submicromolar concentrations we observed to markedly repress ErbB2 transcript levels and others have shown to significantly inhibiting growth of the ErbB2-positive SKBR3, BT-474, and MDA-453 breast cancer cell lines (27). In this regard it must be noted that the NCI/DTP 60 cell line panel, which continues to be used to assess and compare the potential anticancer activity of thousands of synthetic and natural compounds, contains 8 breast cancer cell lines but none of the well characterized ErbB2-positive breast cancer models we have studied (http://dtp.nci.nih.gov/webdata.html). Our findings, therefore, suggest that additional in vitro and in vivo studies are warranted to determine if human breast cancers with ErbB2 amplification and overexpression represent unusually sensitive clinical targets for HDAC inhibitor therapy.

### REFERENCES

1. Benz C C and Tripathy. ErbB2 overexpression in breast cancer: biology and clinical translation. J. Women's Cancer 2: 33-40, 2000.
2. Krauss W C, Park J W, Kirpotin D B, Hong K, Benz C C. Emerging antibody-based HER2(ErbB2/neu) therapeutics. Breast Disease 11: 113-124, 2000.
3. Monia B P, Holmlund J, and Dorr F A. Antisense approaches for the treatment of cancer. Cancer Invest. 18: 635-650, 2000.
4. Roh H, Pippin J, and Drebin J A. Down-regulation of HER2/neu expression induces apoptosis in human cancer cells that overexpress HER2/neu. Cancer Res. 60: 560-565, 2000.
5. Suzuki T, Anderegg B, Ohkawa T, Engebraaten O, Halks-Miller M, Holm P S, Curiel D T, Kashani-Sabet M, and Scanlon K J. Adenovirus-mediated ribozyme targeting of HER2/neu inhibits in vivo growth of breast cancer cells. Gene Ther. 7: 241-248, 2000.
6. Wright L, and Kearney P. Current status of ribozymes as gene therapy agents for cancer. Cancer Invest. 19: 495-509, 2001.
7. Hortobagyi G N, Ueno N T, Xia W, Zhang S, Wolf J K, Putnam J B, Weiden P L, Willey J S, Carey M, Branham D L, Payne J Y, Tucker S D, Bartholomeusz C, Kilbourn R G, DeJager J L, Sneige N, Katz R L, Anklesaria P, Ibrahim N K, Murray J L, Theriault R L, Valero V, Gershenson D M, Bevers M W, Huang L, Lopez-Berestein G, and Hung M-H. Cationic liposome-mediated EIA gene transfer to human breast and ovarian cancer cells and its biologic effects: a phase I clinical trial. J. Clin. Oncology 19: 3422-3433, 2001.
8. Basye J, Trent J O, Gao D, and Ebbinghaus S W. Triplex formation by morpholino oligodeoxyribonucleotides in the HER2/neu promoter requires the pyrimidine motif. Nucleic Acids Res. 29: 4873-4880, 2001.
9. Chiang S-Y, Bürli R W, Benz C C, Gawron L, Scott G K, Dervan P B, Beerman T A. Targeting the Ets binding site of the HER2/neu promoter with pyrrole-imadazole polyamides. J. Biol. Chem. 275: 24246-24254, 2000.
10. Scott G K, Chang C H, Erny K M, Xu F, Fredericks W J, Rauscher III F J, Thor A D, and Benz C C. Ets regulation of the erbB2 promoter. Oncogene 19: 6490-6502, 2000.
11. Benz C C, Scott G K, Sarup J C, Johnson R M, Tripathy D, Coronado E, Shepard H M, and Osborne C K. Estrogen-dependent, tamoxifen-resistant tumorigenic growth of MCF-7 cells transfected with HER2/neu. Breast Cancer Res. Treat. 24: 85-95, 1992.
12. Scott G K, Daniel J C, Xiong X, Maki R A, Kabat D, and Benz C C. Binding of an ETS-related protein within the DNase I hypersensitive site of the HER2/neu promoter in human breast cancer cells. J. Biol. Chem. 269: 19848-19858, 1994.
13. Benz C C, Scott G K, Santos G F, and Smith H S. Expression of c-myc, c-Ha-ras1, and c-ErbB2 proto-oncogenes in normal and malignant human breast epithelial cells. J. Natl. Cancer Inst. 81: 1704-1709, 1989.
14. Chang C-H, Scott G K, Kuo W-L, Xiong X, Suzdaltseva Y, Park J W, Sayre P, Erny K, Collins C, Gray J W, Benz C C. ESX: a structurally unique Ets overexpressed early during human breast tumorigenesis. Oncogene 14: 1617-1622, 1997.
15. Chang C-H, Scott G K, Baldwin M A, Benz C C. Exon 4-encoded acidic domain in the epithelium-restricted Ets factor ESX confers potent transactivating capacity and binds to TATA-binding protein (TBP). Oncogene 24: 3682-3695, 1999.
16. Santos G F, Scott G K, Lee W M F, Liu E, and Benz C. Estrogen-induced post-transcriptional modulation of c-myc proto-oncogene expression in human breast cancer cells. J. Biol. Chem. 263: 9565-9568, 1988.
17. Smith C L and Hager G L. Transcriptional regulation of mammalian genes in vivo: a tale of two templates. J. Biol. Chem. 272: 27493-27496, 1997.
18. Yang X, Ferguson A T, Nass S J, Phillips D L, Butash K A, Wang S M, Herman J G, and Davidson N E. Transcriptional activation of estrogen receptor □ in human breast cancer cells by histone deacetylase inhibition. Cancer Res. 60: 6890-6894, 2000.
19. Wilusz C J, Wormington M, and Peltz S W. The cap-to-tail guide to mRNA turnover. Nat. Rev. Mol. Cell. Biol. 2: 237-246, 2001.
20. Kurokawa H, Lenferink A E G, Simpson J F, Pisacane P I, Sliwkowski M X, Forbes J T, and Arteaga C L. Inhibition of HER2/neu (erbB2) and mitogen-activated protein kinases enhances tamoxifen action against HER2-overexpressing, tamoxifen-resistant breast cancer cells. Cancer Res. 60: 5887-5894, 2000.
21. Park J W, Kirpotin D B, Hong K, Shalaby R, Shao Y, Nielsen U B, Marks J D, Papahadjopoulos D, and Benz C C. Tumor targeting using anti-HER2 immunoliposomes. J. Controlled Release 74: 95-113, 2001.
22. Marks P A, Richon V M, Rifkind R A. Histone deacetylase inhibitors: inducers of differentiation or apoptosis of transformed cells. J. Natl. Cancer Inst. 92: 1210-1216, 2000.
23. Weidle U H, Grossmann A. Inhibition of histone deacetylases: a new strategy to target epigenetic modifications for anticancer treatment. Anticancer Res. 20: 1471-1485, 2000.
24. Yoshida M, Kijima M, Akita M, Beppu T. Potent and specific inhibition of mammalian histone deacetylase both in vivo and in vitro by trichostatin A. J. Biol. Chem. 265: 17171-17179, 1990.
25. Finnin M S, Donigian J R, Cohen A, Richon V M, Rifkind R A, Marks P A, Breslow R, Pavletich N P. Structures of a histone deacetylase homologue bound to the TSA and SAHA inhibitors. Nature 401: 188-193, 1999.
26. Saito A, Yamashita T, Mariko Y, Nosaka Y, Tsuchiya K, Ando T, Suzuki T, Tsuruo T, Nakanishi O. A synthetic inhibitor of histone deacetylase, MS-27-275, with marked in vivo antitumor activity against human tumors. Proc. Natl. Acad. Sci. USA 96: 4592-4597, 1999.
27. Vigushin D M, Ali S, Pace P E, Mirsaidi N, Ito K, Adcock I, and Coombes R C. Trichostatin A is a histone deacetylase inhibitor with potent antitumor activity against breast cancer in vivo. Clin. Cancer Res. 7: 971-976, 2001.
28. Munster P N, Troso-Sandoval T, Rosen N, Rifkind R, Marks P A, and Richon V M. The histone deacetylase inhibitor suberoylanilide hydroxamic acid induces differentiation of human breast cancer cells. Cancer Res. 61: 8492-8497, 2001.
29. Sterner D E, and Berger S L. Acetylation of histones and transcription-related factors. Microbiol. Mol. Biol. Rev. 64: 435-459, 2000.
30. Yao Y-L, Yang W-M, and Seto E. Regulation of transcription factor YYI by acetylation and deacetylation. Mol. Cell. Biol. 21: 5979-5991, 2001.
31. Van Lint C, Emiliani S, and Verdin E. The expression of a small fraction of cellular genes is changed in response to histone hyperacetylation. Gene Expression 5: 245-253, 1996.
32. Mariadason J M, Corner G A, and Augenlicht L H. Genetic reprogramming in pathways of colonic cell maturation induced by short chain fatty acids: comparison with trichostatin A, sulindac, and curcumin and implications for chemoprevention of colon cancer. Cancer Res. 60: 4561-4572, 2001.
33. Scott G K, Marden C, Andrews D, Kirk L, Neve R M, and Benz C C. A genomically integrated ErbB2 promoter-reporting cell system for high-throughput screening and detection of compounds with potential ErbB2 promoter silencing activity. Clin. Cancer Res. 7 (suppl.) abstract #740 from Proc. 2001 AACR-NCI-EORTC Int. Conf., 2001.

### Example 5

### Identification of SNP-1

We identified the existence of a novel SNP in a coding region codon of ErbB2 which substitutes a C for a G nucleotide at cDNA position 3658 resulting in the amino acid substitution of proline (Pro) for alanine (Ala) at amino acid position 1170 (nucleotide and amino acid numbers as oriented by published reference, Coussens et al., Science 230: 1132-1139, 1985). The ErbB2 cDNA sequences flanking and including this G/C polymorphism are as follows: 5′-GAG AGG GCC CTC TGC CTG CTG CCC GAC CTG CT GGT GCC ACT CTG GAA AGG G/CC CAA GAC TCT CTC CCC AGG GAA GAA TGG GGT CGT CAA AGA CGT TTT TGC C-3′ (SEQ ID NO:1).

This SNP was identified from a SNP database search (www.ncbi.nlm.nih.gov/SNP/) where it is referenced as rs1058808 (SNP ID, from 7 sampled chromosomes and human cDNA by handle=LEE; with associated GenBank accession number Hs.173664 M 11730), erroneously listed as being present in an “untranslated region” of the ErbB2 genome. We have correctly identified this as originating from a coding exon in the encoded cytoplasmic domain and c-terminal regulatory region of the ErbB2 receptor tyrosine kinase. In addition to identifying a fraction of the human population with homozygous or heterozygous copies of this SNP-containing allele that may have a different inherited disease risk or likelihood for therapeutic response, the functional consequences of a structure altering Pro substitution in this domain would likely affect ErbB2 receptor function in normal and/or malignant cells, rendering the receptor more or less transforming and tumorigenic, and individuals more or less susceptible to ErbB2-positive tumor formation as well as more or less responsive to anti-ErbB2 therapeutics.

### Example 6

### Identification and Evaluation of SNPs

Example 1 describes our identification of a novel coding region SNP of potential significance in that it produces a structure-altering substitution of Pro for Ala at amino acid 1170 (Ala1170Pro) within the intracellular regulatory region of this oncoprotein and receptor tyrosine kinase known to induce human cancers (ErbB2-positive cancers) and to be a critical target for novel cancer therapeutics (e.g. anti-ErbB2 antibodies and small molecule kinase inhibitors). Using the commercial technique of SnaPshot PCR (ABI) and DNA samples obtained through colleagues at the UCSF Comprehensive Cancer Center, genotyping and sequence detection of this SNP was performed bidirectionally (up to 4× of some troublesome samples) on this collection of normal cell (leukocytes, fibroblasts) and breast tumor DNA samples (see below). Of the batch of human samples initially provided, 66 contained evaluable DNA: 8 normal cell DNA samples from different individuals (fibroblasts or leukocytes) and 58 tumor cell DNA samples from different individuals (50 tumors, 8 tumor cell lines); no matching normal and tumor samples were available. The tumor samples classified as ErbB2-positive (ErbB2+) tumors were distinguished from ErbB2-negative (ErbB2-) tumors by the following definition: of the three ErbB2 assays performed (IHC, TaqMan, CGHcopy#), at least two had to fall within a pre-established ErbB2+ range.

TABLE 2

| | Sample Genotype | | |
|---|---|---|---|
| Sample Type | G/G(Ala) | G/C(Ala/Pro het) | C/C(Pro) |
| normal (n = 8) | 3 | 5 | 0 |
| Tumors (n = 58) | 26 | 21 | 11 |
| ErbB2+ (n = 11) | 3 | 2 | 6 |
| ErbB2− (n = 47) | 23 | 19 | 5 |

TABLE 3

| Allelic frequency: | Ala | Pro |
|---|---|---|
| Normals | 69% | 31% |
| (n = 16 alleles) | (11/16) | (5/16) |
| Tumors | 63% | 37% |
| (n = 116 alleles) | (73/116) | (43/116) |

The public domain genome database contains misannotated and false SNP sequences associated with the ErbB2 genome. One misannotated ErbB2 SNP presently identified as an ErbB2 coding region SNP, Ala1170Pro, is detectable in normal and breast tumor DNA samples. The wildtype Ala allele frequency was found to be 69% in normal cells from different individuals and 63% in cancer (mostly breast) cells from different individuals, with the corresponding Pro allele frequencies of 31% and 37%, respectively (overall 64% Ala frequency and 36% Pro frequency, no significant differences in frequencies between normal and tumor cells). The homozygous Pro encoding genomic variant of ErbB2 appears less prevalent in normal (0/8, 0%) and total breast tumors (11/58, 19%) and most prevalent in the subset of ErbB2+ tumors (6/11, 55%), where its frequency in this cancer subset is 5× higher than in the ErbB2− cancer subset (5/47, 11%). Performing contingency table statistical analyses on the above numbers (chi-square, 2×3) indicates the following: 1) no significant differences in the genotype frequencies between normal and all tumors, or between normal and ErbB2− tumors; and 2) comparing ErbB2+ vs. ErbB2-tumors yields a highly significant difference in the 3 different ErbB2 genotype variants, Ala/Ala, Ala/Pro and Pro/Pro (chi-sq.=11.4, p=0.004).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-56. (canceled)

57. A method of evaluating the responsiveness of a cancer cell to a histone deacetylase (HDAC) inhibitor, said method comprising:
determining whether said cancer cell is a cell comprising amplified or overexpressed ERBB2 wherein a cell that comprises comprising amplified or overexpressed ERBB2 is expected to be more responsive to an HDAC inhibitor than a cell in which ERBB2 is at a normal level.

58. The method of claim 57, wherein an average copy number greater than 1.5 indicates that ERBB2 is amplified.

59. A method of inhibiting the growth or proliferation of a cancer, said method comprising:
determining whether said cancer comprises a cell comprising amplified or overexpressed ERBB2; and
if said cancer comprises a cell comprising amplified or overexpressed ERBB2, contacting cells comprising said cancer with a histone deacetylase inhibitor.

60. The method of claim 59, wherein said contacting comprises contacting said cancer cell with a deacetylase (HDAC) inhibitor in a concentration sufficient to downregulate or silence expression of a HER2/ErbB2/neu oncogene.

61. The method of claim 59, wherein said histone deacetylase (HDAC) inhibitor is selected from the group consisting of trapoxin B and trichostatin A, FR901228 (Depsipeptide), MS-275, sodium butyrate, sodium phenylbutyrate, Scriptaid, M232, MD85, SAHA, TAN-1746, HC-toxin, chlamydocin, WF-3161, Cly-2, NSC #176328 (Ellipticine), 6-(3-aminopropyl)-dihydrochloride, and NSC #321237 (Mercury,(4-aminophenyl)(6-thioguanosinato-N7,S6)-).

62. The method of claim 59, wherein said histone deacetylase (HDAC) inhibitor comprises a hydroxamic acid moiety.

63. The method of claim 59, wherein said deacetylase (HDAC) inhibitor is present in a pharmaceutically acceptable excipient.

64-127. (canceled)
